(19) United States
(12) Patent Application Publication
(10) Pub. No.: US 2013/0211366 A1
(43) Pub. Date: Aug. 15, 2013

(54) INCONTINENCE ARTICLE IN THE FORM OF BRIEFS

(71) Applicants: Oliver Gassner, Ulm (DE); Rüdiger Kessel-Meier, Herbrechtingen (DE); Sandra Max, Heidenheim (DE); Anita Böse, Zierheim (DE); Enno Gause, Heiden-heim (DE)

(72) Inventors: Oliver Gassner, Ulm (DE); Rüdiger Kessel-Meier, Herbrechtingen (DE); Sandra Max, Heidenheim (DE); Anita Böse, Zierheim (DE); Enno Gause, Heiden-heim (DE)

(21) Appl. No.: 13/880,232

(22) PCT Filed: Oct. 14, 2011
(86) PCT No.: PCT/EP2011/005159

§ 371 Date: Apr. 18, 2013

(30) Foreign Application Priority Data
Oct. 19, 2010 (DE) 10 2010 048 932.8

(51) Int. Cl. A61F 13/496

(52) U.S. Cl. CPC A61F 13/4963
USPC 604/385.29

(57) ABSTRACT

The invention relates to an incontinence article (2) in the form of briefs, with a stomach portion (4), a back portion (6) and extending between these in the longitudinal direction (9), a crotch portion (8) which has an absorption body (7) with longitudinal edges (46) and which overlaps the stomach and back portions in the front and rear overlap regions (36, 38) to form a layered structure (13) in the Z-direction (11) and is attached non-releasably thereto in front and rear connection regions (306, 308). The backsheet material (62) and topsheet material (84) surrounding the absorption body extend in the transverse direction (16) outside the longitudinal edges of the absorption body to form an overhang (66a, 66b), which amounts in total to at least 25% relative to a maximum width (E) of the crotch portion. The overhang in each case has first regions (65a, 65a′, 65b, 65b′), which extend inside the overlap regions. The connection regions of the stomach portion and of the back portion comprise second joining regions (314a, 314b, 316a, 316b), which are arranged at least inside the first regions of the overhang, such that the second joining regions form reinforcement regions (334a, 334b, 336a, 336b). In the front or rear overlap region, the first regions have openings (346), which extend through the layered structure in the Z-direction and which form air-permeable regions (350a, 350b, 351a, 351b).

The invention relates to an incontinence article in the form of briefs for receiving body excretions, with a front stomach portion and a rear back portion which, to form a stomach and back band which is continuous in the transverse or waist-encircling direction and has a waist opening that is closed in the waist-encircling direction, are connected to one another at the manufacturer's at side seam regions on both sides, and with a crotch portion, which has an absorbent body, extends in a longitudinal direction between the stomach portion and the back portion and is inseparably joined to the stomach portion and to the back portion in connecting regions, wherein not only the crotch portion but also the stomach portion and the back portion bound the leg openings of the incontinence article. An incontinence article produced in this way from three components is known, for example, from WO 20041052260 A1, WO 03/039423 A1, WO 2005/067842 A1, WO 2005/016200 A1 and EP 1 392 212 B1. In the case of this specific product concept, after the joining of the crotch portion, made to extend in the longitudinal direction, to the stomach portion, made to extend substantially in the transverse or waist-encircling direction, and to the back portion, made to extend correspondingly, in the spread-out flat state, these three components can create an H-shaped basic structure of the incontinence article. The incontinence article is then formed in a modular manner from the components crotch portion, stomach portion and back portion. These components are advantageously first connected to one another by way of the crotch portion, and, preferably, after that the stomach portion is connected to the back portion in side seam regions on both sides. This is a connection performed at the manufacturer's, by which the briefs form is obtained. This connection is typically inseparable. the briefs form may, however, also be separable, in particular along a predetermined breaking line, which may in particular run in the side seam region, for example for removing a used incontinence article from a person needing care.

Incontinence articles in the form of briefs are different in principle from traditional openable and closable incontinence articles in the customary diaper form in that the waist size is generally predetermined and the adaptation to different body sizes is achieved on the basis of a number of standard sizes by an elastic stretchability of the article. Generally used for this purpose are elasticating means, in particular in the form of bands or threads, often referred to as Lycra threads, connected in the pre-stretched state (stretch-bonding process) to chassis materials of the incontinence article, that is to say they are fixed in the pre-stretched state to the chassis materials, for example by means of an adhesive. As a result of their pretensioning, these elasticating means gather the chassis materials together and thereby form pleatings. The incontinence article or the elasticated chassis materials of the incontinence article can then stretch again elastically when the incontinence article is put onto the user like a pair of briefs. Incontinence articles in the form of briefs with such elasticated chassis materials are known in many instances and, for example, are also discussed in the previously mentioned WO 2004/052260 A1.

It has been recognized with the present invention that a three-component incontinence article in the form of briefs formed by the three separate components crotch portion, stomach portion and back portion is exposed to forces during the processes of putting it on and wearing it.

On the one hand, during the process of putting on the incontinence article in the form of briefs, in particular when reaching into the lateral regions of the chassis materials, there is a crumpling, sometimes in certain regions creasing, of the chassis materials provided with elasticating means, accompanied by a twisting of the chassis materials. This represents an impairment of the elasticating effect and consequently of the fit; the visual impression is also disadvantageously impaired.

On the other hand, in a situation where an incontinence article in the form of briefs is being worn, sometimes great forces act, for instance because of the weight of an absorbent body that has been exposed to considerable amounts of liquid. Furthermore, high tensile forces are transmitted within the chassis materials, on the one hand due to the elasticating means that are usually joined onto the chassis materials by a stretch-bonding process, and on the other hand due to tensile forces that are transmitted by movements of the user.

It has been recognized that a three-component incontinence article in the form of briefs that is formed in a modular manner by the connection of the crotch portion to the stomach portion and to the back portion requires a joining connection between these components that is sufficient to meet all the requirements of the intended use, in order to be able to rule out the possibility of the connection between the crotch portion and the stomach portion or the back portion coming undone when great tensile loads occur.

Furthermore, the article should also be reliable and comfortable when worn.

In the case of an incontinence article of the generic type that is made up in a modular manner of the three components the crotch portion, the stomach portion and the back portion, and in particular does not comprise any further components enclosing this modular three-component structure on the outer side of the incontinence article, there is a particular requirement for it to be reliable when worn. This should also be understood as meaning the joining strength of the components in relation to one another, as already stated above due to the influence of forces during the process of putting the article on and in the state of it being worn.

It is also made reliable when being worn by the user by the use of liquid-impermeable backsheet materials, which also serve as clothing protection films.

The use of liquid-impermeable materials as a backsheet material is known. In order to contribute to wearing comfort as well as wearing reliability, it is customary to use liquid-impermeable, but breathable, water-vapor-permeable films as backsheet materials. Nevertheless, even with water-vapor-permeable films, there is the risk of wetting-through. Accordingly, the use of breathable clothing protection films is limited, in particular in the case of incontinence articles with the crotch portion as a component that substantially forms an outer side of the incontinence article.

It has similarly been recognized that, in the case of an incontinence article of the generic type that is formed in a modular manner from the three components the crotch portion, the stomach portion and the back portion, the wearing comfort for the user is reduced on account of the buildup of plies that occurs in the overlapping regions of the components in the Z direction, also including as a result of the two-dimensional stretching of liquid-impermeable backsheet materials, to be specific due to the resultant buildup of heat and accompanying accumulation of perspiration, which apart from discomfort at the time it is being worn can also lead in the long term to irritation of the skin.

Against this background, the present invention is based on the object of countering the problems described above, that is to say in particular forming a stable connection between the crotch portion and the stomach portion and/or between the crotch portion and the back portion in the case of an incontinence article in the form of briefs of the generic type, without this being accompanied by any impairment of the wearing comfort or any other consequences impairing the functionality of the incontinence article.

These objects are achieved according to the invention by an incontinence article with a three-component structure comprising a stomach portion, a back portion and a crotch portion with the features of claim 1.

Advantageous developments of the incontinence article are provided by the respective subclaims.

### DEFINITIONS

The joining region is understood as meaning the areally extending region in which components or plies arranged one on top of the other are connected directly or indirectly by a joining connection extending in the Z direction obtained by joining means.

The Z direction is understood as meaning the direction extending perpendicularly in relation to the plane described by the transverse direction and the longitudinal direction.

The arrangement of the joining means within a first and/or second joining region may in this case be over the full surface area.

The arrangement of the joining means within a first and/or second joining region may also not be provided over the full surface area. Joining means which are not arranged over the full surface area within a joining region may preferably be provided in the form of a punctiform, strip-form or linear arrangement or some other pattern-like arrangement within the joining region. In the case of joining means not arranged over the full surface area, the joining region also comprises the unjoined regions present between the joining means. The association of joining means not arranged over the full surface area with a joining region is determined by a preferably repetitive arrangement of preferably identical joining means with the joining means spaced apart from one another by at most 10 mm. In the case of joining means not arranged over the full surface area, the areal extent of the joining region in the longitudinal and transverse directions is bounded with the aid of an imaginary connecting line through the respectively outer, that is to say distally situated, and neighboring joining means or the outermost peripheral edges thereof.

The reinforcing region formed by the second joining region has the dimensions determined for the second joining region in the longitudinal direction and transverse direction.

“In the region of the absorbent body” is understood here as meaning the positioning of an areal extent that is extended over by the absorbent body in the transverse and longitudinal directions in plan view of an incontinence article laid out flat.

Moreover, “joining region in the region of the absorbent body” describes the arrangement of the joining region in the Z direction, to be specific between the absorbent body and the stomach portion or back portion. Depending on how the components the stomach portion, the back portion and the crotch portion are arranged with their inner and outer sides in relation to one another, the joining region can be further differentiated as “beneath the absorbent body” or “above the absorbent body”. “Joining region beneath the absorbent body” describes the arrangement of the joining region in the Z direction, to be specific between the outer side of the absorbent body and the inner side of the stomach portion or back portion. Analogously, “joining region above the absorbent body” describes the arrangement of the joining region in the Z direction, to be specific between the inner side of the absorbent body and the outer side of the stomach portion or back portion.

“Inner side” of the crotch portion, stomach portion or back portion or of the incontinence article as a whole is understood here as meaning respectively the upper side of the component to be considered or of the incontinence article as a whole that is facing the body of the user. Correspondingly, “outer side” is understood as meaning respectively the upper side of the component to be considered or of the incontinence article as a whole that is facing away from the body of the user, and consequently facing the clothing.

The figures given for lengths and/or widths of the incontinence article as such or of defined regions, such as for example the joining regions and subregions thereof, are always based on dimensions on the incontinence article in its laid-out flat and spread-out flat state.

The openings are provided as a plurality, so that, in their areal distribution, the openings bound “air-permeable regions”. The air-permeable regions comprise the openings and also the regions present between the openings, that is to say non-perforated regions. The association of the openings with an air-permeable region is determined by a preferably repetitive arrangement of preferably identical openings with the openings spaced apart from one another by at most 10 mm. The areal extent of the air-permeable region in the longitudinal and transverse directions is defined with the aid of an imaginary connecting line on the basis of the respectively outer, that is to say distally situated, and neighboring openings, to be specific at the outermost peripheral edges thereof.

“Overhang” is understood here as meaning the extent of the backsheet material or of the topsheet material and the backsheet material in the transverse direction laterally outside the longitudinal peripheries of the absorbent body, the maximum extent being used in each case, that is to say the outer extent of the topsheet material and/or the backsheet material situated distally furthest from the longitudinal peripheries of the absorbent body. The backsheet material and/or the topsheet material may advantageously consist of a number of components, thus for example the topsheet material may advantageously be a composite of a topsheet material and barrier means adjacent on both sides in the longitudinal direction, such as for example the so-called cuffs. It is therefore understood that, even in the case of composites, that is to say composed topsheet materials and/or backsheet materials in which the individual plies do not cover one another congruently, when considering the overhang, the maximum outer extent of the composite, or of the individual material plies occurring therein, that is to say the outer extent situated distally furthest away, is used in each case. The overhang is respectively assigned the width H.

The overhang has in this case “first regions” and “second regions”: A first region respectively extends on both sides of the longitudinal peripheries of the absorbent body in the front and rear overlapping regions. A respective second region extends on both sides of the longitudinal peripheries of the absorbent body between the transverse peripheries of the stomach portion and of the back portion that are facing the crotch, that is to say outside the overlapping regions.

“Side region of the stomach portion and/or back portion” is understood here as meaning the extent of the chassis material of the stomach portion and/or back portion laterally outside the respective longitudinal periphery of the crotch portion in the transverse direction to the respective longitudinal peripheral portion of the stomach portion or back portion. In this case, as explained above, the maximum extent, that is to say the outer extent that is situated distally furthest away, of the topsheet material and/or of the backsheet material is used as the longitudinal periphery of the crotch portion. The side region of the stomach portion and/or of the back portion is respectively assigned the maximum possible width N. Also in the case where the crotch-side portion of the stomach portion or of the back portion that is facing the leg openings has a peripheral contour deviating from the transverse or waist-encircling direction and running in the direction of a transverse center axis of the crotch portion, thus in particular if this peripheral contour is arcuately shaped, the maximum width N is always used. In other words, the width N is always the distance measured in the transverse direction from the longitudinal periphery of the crotch portion to the longitudinal periphery of the stomach portion or back portion, which then forms the side seam regions of the incontinence article.

With the invention it has been recognized that such a construction of an incontinence article in the form of briefs is accompanied by major advantages:

A great overhang of the backsheet material or of the topsheet material and backsheet material in the transverse direction respectively outside the longitudinal peripheries of the absorbent body is accompanied by the advantage that the crotch portion can be securely fixed to the back portion and the stomach portion at the manufacturer's on account of the great overlapping region of the crotch portion and the stomach portion and of the crotch portion and the back portion.

The formation of a second joining region in the first region of the overhang that is arranged within the overlapping region has the effect of forming as it were reinforcing regions. This has the advantage that the first and/or second elasticating means are arranged, at least in a subregion, between two stiffened panels extending in a longitudinal direction, which run substantially transversely, at least with a significant component in the transverse direction, to the first and/or second elasticating means. While one reinforcing region forms a first stiffened panel, the side seam usually represents a second stiffened panel. This has a positive evening-out effect on the distribution of the restoring forces of the first and/or second elasticating means gathering the chassis materials in this subregion. As a consequence, very uniform gathering can take place there and the undesired pleatings of the chassis materials, and accompanying uncontrolled twisting of the chassis materials, can be counteracted. This consequently has a positive effect on the fit of the incontinence article.

The fact that the connecting regions of the crotch portion to the stomach portion and to the back portion correspondingly comprise in each case first and second joining regions also makes it possible for a connection between the crotch portion and the stomach portion or back portion to be configured and designed appropriately for the requirements of the respective regions of the incontinence article: It has been recognized that the absorbent body is stabilized in its positioning within the incontinence article, in particular with respect to the flexible chassis materials of the stomach and back portions, by the first joining region, which runs at least in the region of the absorbent body. This is advantageous since, during the process of putting on an incontinence article in the form of briefs, which differs considerably from the process of putting on a traditional diaper of the open type, forces act on the entire incontinence article. While the diaper of the open type is correspondingly put flat onto the body of the user, without any great tensile forces acting, and is then subsequently closed, when putting on the briefs-type diaper that is closed at the manufacturer's, for example when pulling it up between the legs, the absorbent body and the plies neighboring it are exposed to deforming forces that are anything but inconsiderable. A disadvantageous deformation, and possibly permanent significant displacement, of the crotch portion in relation to the chassis materials of the stomach and back portions is prevented by the attachment at least in certain portions in the region of the absorbent body.

Even though, as stated above, an overhang is considered to be positively necessary for a stable assembly of the three-component incontinence article, it has on the other hand been recognized that a buildup of heat, and consequently also of perspiration, occurs in the region of the overlap on account of the large number of plies in comparison with the region outside the contour of the crotch portion. The buildup of plies in the overlapping region of the overhang in this case comprises the backsheet material, which does on the one hand make the article more reliable for the user to wear by preventing bodily fluid from running out. On the other hand, the liquid-impermeable property of the clothing protection film, disadvantageously accompanied by the accumulation of heat, and consequently perspiration, comes into play much more in the region of the overhang than in the overlapping regions, in which the absorbent body also extends: The property of absorbing bodily fluids that is an inherent feature of the absorbent body has the effect that in this region there is the possibility of soaking up accumulations of perspiration caused by the buildup of plies, and also the consequent sealing effect. By contrast, the dominant property in the overhang extending on both sides of the longitudinal peripheries of the absorbent body is the sealing of the clothing protection film, almost right next to the skin of the user, without the such strongly soaking-up capacities of the absorbent body, while on the other hand an overly great areal extent of the absorbent body is also only desirable to a certain degree, on account of costs and also a visually adverse fit due to the bulkiness of the absorbent body. The provision of openings extending through the buildup of plies in the Z direction creates air-permeable zones in the first region of the overhang, which has positive effects on the wearing comfort of the incontinence article.

The features of patent claim 1 altogether provide an incontinence article in the form of briefs with the three-component structure mentioned, in which a secure connection of the components can be realized and the described disadvantages of the prior art are overcome, to be precise without involving any impairment of the wearing comfort or the functionality of the incontinence article or its components, but instead significantly improving the fit of the incontinence article as well as the wearing comfort and wearing reliability.

The openings according to the invention that extend through the buildup of plies in the Z direction are provided in the first regions of the overhang in the front or rear overlapping region.

In particular in the front overlapping region, the first region of the overhang advantageously has the openings that extend through the buildup of plies in the Z direction: In the state in which the incontinence article is used and worn, the overhang extending in the transverse direction in the front overlapping region reaches over the parts of the body located in or around the pubic region and areas adjacent thereto. These regions of the body in particular are known as perspiration zones. This is so especially because, in the state in which the article is worn, instances of the skin folding over between the upper leg and where it joins the pelvis that occur for example during sitting can cause buildups of heat, and consequently accumulations of perspiration, in these regions. Openings in the first region of the overhang that are located at or near the regions of the incontinence article adjoining the leg openings as a result of the modular three-component structure of the incontinence article consequently contribute positively to the wearing comfort.

Depending on the application area of the incontinence article and also the mobility of the user, it may be advantageous to design the first regions of the overhang differently in the front and rear connecting regions. For instance, the openings in the first region of the overhang could also be provided in particular only in the rear overlapping region. For users of incontinence articles in the form of briefs with less mobility, that is to say in the case of bedridden persons, the rear overlapping region is subjected to particular loading on account of the user predominantly lying on the rear side. The advantages and disadvantages explained above occur here particularly in the rear overlapping region, such as indeed the stable connection of the components in relation to one another that is made possible by the overhang, but on the other hand the disadvantageous buildups of heat and accumulations of perspiration due to the buildup of plies. Also in this respect, the wearing comfort can be increased by the openings provided in the rear overlapping region in the first region of the overhang.

In a development of the invention, it proves to be particularly advantageous if, both in the front overlapping region and in the rear overlapping region, the first regions of the overhang have openings that extend through the buildup of plies in the Z direction. This allows an incontinence article that is independent of the later area of use, and consequently can be used universally and meets different sets of requirements, to be provided.

The incontinence article is formed in particular in a three-part modular manner, comprising the separate components crotch portion, stomach portion and back portion, i.e., apart from these three components, the incontinence article in the form of briefs does not include any further components surrounding this modular three-component structure in the form of briefs in its entirety, that is to say enclosing it. This, however, is based on the understanding that it does not rule out the possibility that the three components as such, that is the crotch portion, stomach portion and back portion, may comprise a variety of subcomponents, plies or materials.

The components respectively have an inner side and an outer side. In this case, the joining connections between the components may be oriented differently in relation to one another.

In the respective connecting regions, the crotch portion may be undetachable joined with its outer side onto the inner side of the stomach portion and/or onto the inner side of the back portion. If the crotch portion is joined with its outer side onto the respective inner side of the stomach portion or back portion, the stomach portion or back portion can, as a component that is continuous in the transverse direction, perform a function of enclosing, and consequently fixing, the longitudinal ends and the regions adjacent thereto of the crotch portion in the overlapping region, which consequently has positive effects on wearing comfort and wearing reliability.

In an alternative configuration, in the respective connecting regions the crotch portion may be undetachably joined with its inner side onto the outer side of the stomach portion and/or onto the outer side of the back portion. As a result of the joining of the crotch portion with its inner side to the respective outer side of the stomach portion or back portion, the stomach portion or back portion lies directly against the body of the user as a component that is continuous in the transverse direction. This is advantageous because it means that the bulkiness introduced to a certain degree by the crotch portion, in particular by the absorbent body extending in the longitudinal direction, and also the quite strong, sudden change in properties abruptly occurring at the longitudinal peripheries of the crotch portion or the absorbent body due to the structural design, and consequently inhomogeneity in the stomach or back region, is no longer felt directly by the user, but instead in this respect the incontinence article rather gives the user the impression in the stomach or back region of wearing a normal pair of underwear briefs.

In a further configuration, the crotch portion may be different in the two overlapping regions, and consequently in the front and rear connecting regions, that is to say be undetachably joined on the one hand with its inner side and on the other hand with its outer side to the stomach portion or back portion.

For the first and second joining regions, non-adhesive and/or adhesive joining means or combinations thereof may be advantageously used.

Non-adhesive joining means are, in particular, taken from the group of welding locations, more particularly ultrasonic welding locations, thermal welding locations and/or calender welding locations.

For adhesive joining means, backing-bonded adhesives, such as adhesive tapes, in particular double-sided adhesive tapes, and/or non-backing-bonded adhesives may be used. In particular, hotmelt adhesive is used as the adhesive. More particularly, the adhesive has hydrophobic properties.

The following for example may be preferably used as adhesives: D 9105 ZP or LC 3001 ZP (H.B. Fuller Deutschland GmbH, An der Roten Bleiche 2-3, 21335 Lüneburg, Germany); H20028 or H 2481 (Bostik Nederland B.V., Zeggeveld 10, 4705 RP Roosendaal, the Netherlands); Technomelt Q2415 or Technomelt Q5430 (Henkel KGaA, 40191 Düsseldorf, Germany).

The arrangement of the joining means within a first and/or second joining region may in this case be over the full surface area or not over the full surface area.

It has moreover proven to be particularly advantageous to use an adhesive, particularly a hotmelt adhesive, as joining means in a first joining region. More particularly, the adhesive or hotmelt adhesive has hydrophobic properties. This is advantageous since, in addition to the connecting function, a liquid barrier is formed at the same time.

In the first joining region, the crotch portion may advantageously be connected to the stomach portion and/or to the back portion by means of an application of adhesive that is not over the full surface area. An application of adhesive that is not over the full surface area may be, for example, a strip-form pattern, a web-form continuous or discontinuous grid structure or insular regions or else a strip-form or spirally arranged adhesive structure. It has alternatively proven to be particularly advantageous to use in the first joining region an application of adhesive over the full surface area. The first joining region provides optimized attachment of the crotch portion, with an absorbent body that is in itself torsionally stiff, to the rather more flexible chassis materials of the stomach portion and/or back portion and prevents undesired displacement of the components in relation to one another.

Advantageously, the first joining region extends in the region of the absorbent body at least up to the longitudinal peripheries of the absorbent body, but ends before the longitudinal peripheries of the crotch portion. More advantageously, the first joining region extends in the transverse direction over the longitudinal peripheries of the absorbent body in such a way that an overlap with the respective second joining region is obtained.

Depending on the arrangement of the crotch portion and the stomach portion or back portion with their inner and outer sides in relation to one another, the first joining region lies beneath or above the absorbent body.

The applications of adhesive described above, in particular applications of adhesive not over the full surface area, may similarly be used for the second joining region.

Non-adhesive joining means may be used particularly advantageously for a second joining region. The forming of a second joining region with joining means in the form of welding locations, in particular ultrasonic welding locations, thermal welding locations and/or calender welding locations, is advantageous: The use of adhesive materials also gives rise to the problem when the incontinence articles are produced by machine that extremely precise positioning of the adhesive material is required, but not readily achievable in high-speed diaper machines, in order to prevent the adhesive materials from escaping over the peripheral region, and consequently prevent the sticking together of regions and materials not intended for this within the incontinence article or between a number of incontinence articles. The escape of adhesives also leads to contamination of the tools of the machine during the production of the incontinence article. The use of joining means in the form of welding locations has the effect of avoiding the unwanted escape of adhesive material caused by the production equipment. Moreover, the joining means in the form of welding locations ultimately provide a reinforcing region without introducing further additional material components, which saves costs.

Depending on the application area of the incontinence article and also the mobility of the user, it may be advantageous to design the second joining regions differently in the front and rear connecting regions. Preferably, the second joining regions in the front and rear connecting regions differ at least in one of the parameters longitudinal extent, width, degree of overlap, type of joining means and/or arrangement of the joining means and/or combinations thereof.

Depending on the area of use of the incontinence article and also the mobility of the user, it may be advantageous to design the second joining regions differently in the longitudinal extent in the front and rear connecting regions. More preferably, the second joining regions have a greater longitudinal extent in the rear connecting region than in the front connecting region.

It is essential for the invention that openings extending through the buildup of plies in the Z direction are provided in the first region of the overhang. The openings may in this case be introduced in various ways. For instance, the openings may be introduced by means of perforating measures, such as by a cutting process or by punching out in the existing buildup of plies. In an alternative measure, the openings may be introduced by means of welding locations extending through the buildup of plies in the Z direction. It is also conceivable to use the measures in combination.

In a particularly advantageous way, the openings are formed by joining means, in particular in the form of welding locations, more particularly ultrasonic welding locations, thermal welding locations and/or calender welding locations. The introduction of the openings by means of joining means, in particular welding locations, advantageously allows a joining connection of the overhang to the stomach portion and/or back portion, and consequently also a reinforcing region and the air permeability that is conducive to wearing comfort, to be provided at the same time in a single method step in a technically simple manner.

It is also advantageous if the openings extending through the buildup of plies in the Z direction are arranged at least within the second joining regions. In joining regions, a joining connection, extending in the Z direction, between the components or plies arranged one on top of the other is directly or indirectly provided by definition in the Z direction, the Z direction being understood as meaning the direction that extends perpendicularly to the plane described by the transverse direction and the longitudinal direction. The joining regions accordingly have the effect that the plies/layers are arranged in closer proximity to one another, and consequently the sealing effect is also increased, and with it the buildup of heat and perspiration. It is all the more advantages to arrange the openings extending through the buildup of plies within the second joining region.

In an advantageous way, the second joining regions and the air-permeable regions coincide in their areal extent, in particular are disposed congruently one on top of the other. This allows the synergistic effect of the joining connection and the wearing-comfort-enhancing air permeability to be provided advantageously for the user of the incontinence article within the second joining regions. In particular, it may be provided that a second joining region and an air-permeable region are disposed congruently one on top of the other in their areal extent. This is achieved particularly advantageously if the second joining region and the air-permeable region are introduced into the incontinence article in the form of briefs at the same time in one method step, such as in particular by introducing welding locations, such as in particular ultrasonic welding locations, thermal welding locations and/or calender welding locations.

In a development of the invention, it proves to be advantageous if, from the crotch-facing transverse periphery of the stomach portion and/or the back portion, when considered in the longitudinal direction, a second joining region, and consequently a reinforcing region and/or an air-permeable region, extends in the direction of the longitudinal ends of the crotch portion, in particular continuously up to at least the respective longitudinal end of the crotch portion. Providing the second joining regions, and consequently the reinforcing regions, from the crotch-facing transverse periphery of the stomach portion and/or back portion has a counteracting effect already from the regions which bound, and consequently form, the leg openings and which, as distal regions, are also exposed more to the problem of creasing, and consequently undesired twisting, of the chassis materials. Providing the air-permeable regions from the crotch-facing transverse periphery of the stomach portion and/or back portion has the effect of providing regions that are conducive to wearing comfort already from the regions which bound, and consequently form, the leg openings and which, as explained above, are also zones where perspiration collects.

In another advantageous way, the second joining region extends over a subregion of the overhang in the transverse direction with a width P′ and/or the air-permeable region extends over a subregion of the overhang in the transverse direction with a width M′. This gives the second joining region and/or the air-permeable region a defined extent in the transverse direction. Preferably, the second joining region or the air-permeable region extends over a respective subregion of the overhang in the transverse direction in a continuously constant width P′ or M′. This is also advantageous in particular for the technical way in which a respective region is provided within the incontinence article during production.

In a further advantageous way, the second joining region ends at a distance AP before the longitudinal periphery of the absorbent body. Depending on the type of joining means used within the second joining region, it may be advantageous to end the second joining region at a distance AP from the absorbent body. Thus, it is advantageous for example when using non-adhesive joining means, such as welding locations, to use them such that they do not extend into the region of the absorbent body, in order in this way to avoid the instances of compacting or even openings that are disadvantageous for a peripheral region of the absorbent body and may adversely affect and even minimize the absorption capacity. More particularly, the second joining region is arranged from the longitudinal periphery of the crotch portion. The arrangement of the second joining region from the longitudinal periphery of the crotch portion advantageously avoids unattached side peripheries of the overhang, which in their unattached state may disadvantageously lead to instances of creasing, and consequently also irritation of the skin.

The distance AP is preferably at least 3 mm, more preferably at least 5 mm, more preferably at least 7 mm, more preferably at least 10 mm, more preferably at most 40 mm, more preferably at most 30 mm, more preferably at most 20 mm.

In a further advantageous way, the air-permeable region ends at a distance AM before the longitudinal periphery of the absorbent body. Providing a distance between the openings extending through the buildup of plies in the Z direction and the absorbent body advantageously ensures that the advantages brought about by the openings with regard to reducing buildups of heat and accumulations of perspiration are not disadvantageously accompanied by a reduced barrier function, and consequently a reduced or adversely affected escape preventing function. In the peripheral region of the absorbent body there is the risk of absorbed bodily fluids escaping by being forced out. Similarly, in configurations in which barrier means, known as cuffs, are provided along or in the region of the longitudinal periphery of the absorbent body, a possible impairment of these barrier means by openings unintentionally introduced therein, and consequently reduction of their barrier function, is avoided. More particularly, the air-permeable region is arranged from the longitudinal periphery of the crotch portion.

The distance AM is preferably at least 3 mm, more preferably at least 5 mm, more preferably at least 7 mm, more preferably at least 10 mm, more preferably at most 40 mm, more preferably at most 30 mm, more preferably at most 20 mm.

The distance AP and the distance AM are understood in this case as being measured in the transverse direction.

In a preferred way, the distance AM, measured in the transverse direction, corresponds to the distance between the longitudinal periphery of the absorbent body and the leg elasticating means introduced in particular in the second region of the overhang, measured in the transverse direction.

The second joining regions provided in the front and/or rear connecting region are preferably in each case arranged symmetrically to one another and preferably also have in each case the same design, so that they therefore coincide, for example, with regard to longitudinal extent, width, degree of overlap, type of joining means and/or arrangement of the joining means and/or combinations thereof.

In an advantageous way, the second joining region has in the extended-over subregion of the overhang in the transverse direction a width P′ of greater than 1 mm, preferably greater than 2 mm, more preferably greater than 5 mm, but preferably of less than 60 mm, preferably less than 50 mm, more preferably less than 40 mm, more preferably less than 30 mm, more preferably less than 20 mm.

In a further advantageous way, the proportion P′/H of the subregion of the respective overhang that is extended over in the transverse direction by the second joining region, with the width P′ with respect to the respective overhang with a width H in the front and/or rear overlapping region, is at least 0.01, particularly at least 0.04, more particularly at least 0.07, more particularly at least 0.10, but preferably at most 0.90, particularly at most 0.80, more particularly at most 0.70, more particularly at most 0.60, more particularly at most 0.50, more particularly at most 0.40. This proportion of the joining region in the respective overhang allows adequate reinforcement of the attachment of the chassis-forming shell materials to the crotch portion to be achieved, without the flexibility of the overhang being impaired to any considerable extent.

In an advantageous way, the respective overhang has a width H preferably of at least 10 mm, more particularly of at least 20 mm, more particularly of 20 to 100 mm, more particularly of 20 to 80 mm.

In an advantageous way, the proportion P′/N of the respective subregion of the respective overhang that is extended over in the transverse direction by the second joining region, with the width P′ with respect to the respective side region of the stomach portion and/or of the back portion with a width N, is at least 0.01, particularly at least 0.015, more particularly at least 0.020, but preferably at most 0.35, particularly at most 0.30, more particularly at most 0.25, more particularly at most 0.20, more particularly at most 0.15, more particularly at most 0.10.

In an advantageous way, a respective side region of the stomach portion and/or of the back portion extends in the transverse direction with a width N of preferably at least 100 mm, more preferably of at least 120 mm, and particularly 120 mm to 350 mm, more particularly 120 to 320 mm.

The air-permeable regions provided in the front and/or rear connecting region are preferably in each case arranged symmetrically to one another and preferably also have in each case the same design, so that they therefore coincide, for example, with regard to longitudinal extent, width, type of openings and/or arrangement of the openings and/or combinations thereof.

In an advantageous way, the air-permeable region has in the extended-over subregion of the overhang in the transverse direction a width M′ of greater than 1 mm, preferably greater than 2 mm, more preferably greater than 5 mm, but preferably of less than 60 mm, preferably less than 50 mm, more preferably less than 40 mm, more preferably less than 30 mm, more preferably less than 20 mm.

In a further advantageous way, the proportion M′/H of the subregion of the respective overhang that is extended over in the transverse direction by an air-permeable region, with a width M′ with respect to the respective overhang with a width H in the front and/or rear overlapping region, is at least 0.01, particularly at least 0.04, more particularly at least 0.07, more particularly at least 0.10, but preferably at most 0.90, particularly at most 0.80, more particularly at most 0.70, more particularly at most 0.60, more particularly at most 0.50, more particularly at most 0.40.

In an advantageous way, the proportion M′/N of the respective subregion of the respective overhang that is extended over in the transverse direction by an air-permeable region, with the width M′ with respect to the respective side region of the stomach portion and/or of the back portion with a width N, is at least 0.01, particularly at least 0.015, more particularly at least 0.020, but preferably at most 0.35, particularly at most 0.30, more particularly at most 0.25, more particularly at most 0.20, more particularly at most 0.15, more particularly at most 0.10.

In a further advantageous development, the joining means in the second joining region are not provided over the full surface area. Particularly, the joining means within the second joining region are provided in the form of a pattern, particularly in the form of a punctiform and/or strip-form and/or linear arrangement and/or some other pattern-like arrangement and/or in combinations thereof. As a result, the extent of the stiffening of the second joining regions can be advantageously set. As described above, the stiffening has an advantageous effect on the fit; however, excessive stiffening may lead to uncomfortable hard locations.

In a further advantageous way, the sum of the joining means not arranged over the full surface area in the second joining region assumes an attached surface area (joining locations) with a proportion of at least 1.5%, particularly at least 2.0%, more particularly at least 2.5% and preferably of at most 60%, more particularly at most 50%, more particularly at most 40%, more particularly at most 30%, more particularly at most 20%, with respect to the overall surface area extended over by the second joining region.

In a particularly advantageous way, the joining means in the second joining region are arranged in a point pattern and the sum of the surface area attached by the joining means (joining locations) assumes a proportion of at least 1.5%, particularly at least 2.0%, more particularly at least 2.3%, more particularly at least 2.5%, and preferably at most 20.0%, particularly at most 15%, more particularly at most 10.0%, more particularly at most 8.0%, more particularly at most 7.0%, more particularly at most 6.0%, with respect to the overall surface area extended over by the second joining region.

In a further advantageous way, the surface areas attached by the individual joining means in a point pattern in the second joining region have a diameter of at least 0.2 mm, particularly of at least 0.3 mm, more particularly of at least 0.4 mm, more particularly of at least 0.5 mm and preferably of at most 2.5 mm, particularly of at most 2.0 mm, more particularly of at most 1.5 mm, more particularly of at most 1.2 mm, more particularly of at most 1.0 mm.

In particular, the neighboring individual joining means present in a point pattern are respectively arranged spaced apart from one another by 1-10 mm, particularly by 1-8 mm, more particularly by 1-6 mm, more particularly by 1-5 mm, more particularly by 1.5-4.5 mm, more particularly by 2-4 mm, particularly preferably at the same distance.

In a further advantageous way, the sum of the openings arranged in the air-permeable region assumes a surface area extended over by the openings with a proportion of at least 1.5%, particularly at least 2.0%, more particularly at least 2.5% and preferably of at most 60%, particularly at most 50%, more particularly at most 40%, more particularly at most 30%, more particularly at most 20%, with respect to the overall surface area extended over by the air-permeable region.

In a particularly advantageous way, the openings are arranged in a point pattern and the sum of the surface area extended over by the openings assumes a proportion of at least 1.5%, particularly at least 2.0%, more particularly at least 2.3%, more particularly at least 2.5%, and preferably at most 20.0%, particularly at most 15%, more particularly at most 10.0%, more particularly at most 8.0%, more particularly at most 7.0%, more particularly at most 6.0%, with respect to the overall surface area extended over by an air-permeable region, in particular with respect to the overall surface area extended over by a second joining region. This proportion of openings within an air-permeable region, in particular within a second joining region, allows an adequate air permeability to be achieved in the overlapping region, without the material damage that is in fact introduced there to a certain extent by the openings impairing the properties of the materials used, such as for example tearing and stretching resistances, to any considerable extent.

Particularly advantageously, the openings extending through the buildup of plies in the Z direction have a diameter of at least 0.2 mm, particularly of at least 0.3 mm, more particularly of at least 0.4 mm, more particularly of at least 0.5 mm and preferably of at most 2.5 mm, particularly of at most 2.0 mm, more particularly of at most 1.5 mm, more particularly of at most 1.2 mm, more particularly of at most 1.0 mm. Introducing openings of relatively small dimensions avoids excessive perforation of the components or plies. On the one hand, openings of large dimensions are indeed advantageous for increased air permeability. On the other hand, openings of large dimensions represent a potential for damage to the material plies or components, particularly when tensile forces occurring during the state in which the incontinence article is put on and worn are acting.

In particular, the neighboring individual openings present in a point pattern are respectively arranged spaced apart from one another by 1-10 mm, particularly by 1-8 mm, more particularly by 1-6 mm, more particularly by 1-5 mm, more particularly by 1.5-4.5 mm, more particularly by 2-4 mm, particularly preferably at the same distance.

In particular, the air-permeable region has an air permeability which is greater than the air permeability in the first region of the overhang outside the air-permeable region by a factor of 1.5, particularly 2, particularly 2.5, more particularly 3, more particularly at most 5, more particularly at most 4.5.

Apart from the air permeability, the introduction of openings also influences the water vapor permeability, in the sense that, when there is air permeability there is also water vapor permeability.

In particular, the air-permeable region has a water vapor permeability which is greater than the water vapor permeability in the first region of the overhang outside the air-permeable region by a factor of 1.5, particularly 2, particularly 2.5, more particularly 3, more particularly at most 5, more particularly at most 4.5.

The determination of the air permeability is performed in this case as follows:

The measurement of the air permeability is based on the standard DIN EN ISO 9237: 1995. The test method serves for assessing the air permeability of textile sheet-like formations. In this case, the air permeability is a characteristic value for describing the properties of textile sheet-like formations for allowing air to flow through under given differential pressure.

The air permeability is expressed as the speed of an air stream that passes through the sample being measured, perpendicularly to the surface under fixed conditions, to be specific for the test area, the differential pressure and the time.

### Testing Apparatus:

An air permeability tester in accordance with DIN EN ISO 9237 should be used as the testing apparatus. Such an air permeability tester comprises a circular sample holder having an is opening with a defined test area, such as for example of 20 cm², also a device for fastening the sample being measured securely and without twisting, more preferably also in addition a protective ring device in addition to the aforementioned device, for preventing air from escaping over the edges of the sample, and also a pressure gage connected to the testing head, a device for producing a constant air flow and for setting the flow rate with which a differential pressure can be produced, and furthermore a flowmeter for indicating the flow rate.

The apparatus of the type FX 3300 Labortester IIII from the company Textest AG, Schwerzenbach, Switzerland, may be used for example for carrying out the measurement.

### Sample Preparation:

For sample preparation, the sample must be kept in a standard conditioning atmosphere of 23° C./50% relative humidity for at least 24 hours before the testing begins.

### Test Procedure:

With respect to the test procedure, it must be ensured that, in comparable investigations, the tests must be carried out under the same conditions, with the same test area and the same differential pressure. The sample being measured must be fastened on the circular sample holder with adequate tension to avoid folding. If folds occur, however, it must be ensured that the sheet-like formation, that is to say the test sample, is not twisted in the clamping plane. At the same time, however, selvages and areas with crumpling or folded locations must generally be avoided. In the case of sheet-like formations, in which the air permeability may be different in the two possible testing directions, it must be recorded which side has been tested. In the case of textile sheet-like formations that are coated on one side, the coated side should be clamped in the direction of the negative pressure side, in order to avoid leaks. The suction blower suitable for forcing the air through the textile sheet-like formation or other such device should be put into operation and the flow rate for achieving the differential pressure should be set infinitely variably. After achieving flow rates under stable conditions, at least after waiting for a least one minute, the flow rate should be noted. The testing should be repeated at least 10 times under the same conditions at different locations of the sample being measured. 100 Pa or 200 Pa is recommended as the differential pressure. The differential pressure used for the testing should be specified.

### Evaluation:

The air permeability R should be calculated in mm/s with the aid of the equation specified in the standard

$$R = \frac{q ( v )}{A} \times 167$$

or in m/s on the basis of the equation

$$R = \frac{q ( v )}{A} \times 0.167$$

where
q (v): is the arithmetic mean of the air flow in dm³/min (l/min)
A: is the test area, in cm²
167: is the conversion factor of dm³/min or l/min per cm², in mm/s
0.167: is the conversion factor of dm³/min or l/min per cm², in m/s

### As a Modification of the Standard DIN EN ISO 9237: 1995:

In the case of investigations in which no test sample adapted to the test area of the circular sample holder is available or can be provided, such as for example in the case of relatively small and/or non-circular test samples, a test sample obtained by means of combining with a backing material may be used.

### Producing the Test Sample:

Providing a test piece from the material to be investigated with defined dimensions, for example 50×10 mm.
Preparing a defined air- and water-impermeable backing material and punching out a round piece, which is adapted to the presented circular sample holder with a defined test area. A film, PE film HyFol PP Plus, 16 μm from RKW Wasserburg, Germany, may be used for example as the backing material.
Punching out a part from the backing material, this punch-out being of a slightly smaller size in comparison with the test piece punched out from the material under investigation, so that the test piece can be fastened well in the region of the punched-out hole of the backing material. For example, 45×7 mm with respect to the aforementioned application example of the test piece.
Affixing of the test piece into the backing material by means of a water- and air-impermeable adhesive tape, to be precise on both sides and with a small overlap, in order that no air can flow through between the backing material and the test piece. For example, crystal clear Tesafilm®, 15 mm×10 m from the company Tesa SE may be used for this. The surface area remaining for the air stream to pass through after the test piece has been fastened, that is to say the surface area of the test piece that is not covered by adhesive tape on either one or the other side, should be noted in its dimensions.

### Measurement:

The measurement is carried out on the basis of the test procedure specified in the method, it also being necessary in particular to carry out measurements that are necessary for correction and standardization, known as negative and zero controls, at the same time as and along with the measurement of the actual test sample for the backing material and for the materials introduced for fastening:
a) Measurement of the zero control: For this purpose, the intact backing material as such, that is to say still without the punched-out receiving region for the test piece, is measured.
b) Measurement of the negative control: For this purpose, the backing material with the punched-out region, which is just covered over with the adhesive tape, is measured. Here, the permeability of the adhesive tape and the seam produced therewith is measured and checked.
c) Measurement of the test sample produced as described above.

### Evaluation:

The measured value of the test sample is standardized to the zero control of the test piece and then reduced by the value of the negative control.
Apart from the units specified in the standard of mm/s or m/s, the air permeability may also be specified in some other units, such as l/m²/min or l/m²/s.

The determination of the water vapor permeability is performed in this case as follows:

The measurement of the water vapor permeability is based on the standard ASTM E 96-0, applying the water method variant: A testing temperature of 37° C. is used.

The test procedure serves for determining the water vapor permeability of sheet-like formations, such as for example textiles, breathable films or film composites. In this case, the material property of transporting water vapor away from the side of the material near the body to the side of the material away from the body is determined.

### Required Testing Apparatus/Reagents:

Conditioning cabinet for constant atmosphere: 37±1° C., 50±2% relative humidity
Steel testing cup with an annular cover, silicone sealing ring and fixing screws, having a defined upper opening, the open cross section having a diameter of 62 mm, which corresponds to an effective test area of 0.00302 m². The entire testing cup has all the way round an inside diameter of 62 mm and has a height of 55 mm.
Round punching blade with a diameter of 90 mm
Balance
Measuring rod with millimeter division
Distilled water
Petroleum spirit, boiling point 60-80° C.

### Sample Preparation:

If the sheet-like material to be tested is not available as the desired raw material, but in a composite, such as for example in a sanitary product, petroleum spirit is used to detach it. The material is covered with petroleum spirit in a covered pan and left to stand for approximately 20 min. Then, the material is lifted out of the pan and the undesired attached residual materials should easily allow themselves to be stripped off. The solvent is then allowed to evaporate.

In connection with the investigation of the water vapor permeability of the buildup of plies in the Z direction in the incontinence article in the form of briefs according to the invention, the composite/buildup of plies as such is introduced into the test procedure.

For sample preparation, the sample must be kept in a standard conditioning atmosphere of 23° C./50% relative humidity for at least 24 hours before the testing begins. The test samples are prepared by punching out the test pieces from the material to be tested, to be precise in adapted size, so that the test sample can cover the opening of the steel testing cup completely and without any folds. The steel testing cup and the water to be placed therein are preheated to 37° C. The steel testing cup is filled with the distilled water, to be precise in such way as to leave a distance of 30±2 mm from the upper rim of the testing cup. Subsequently, the test sample is placed onto the rim of the testing cup, with the side of the test sample that is away from the body facing upward, the sealing ring and the annular cover are placed on top and the fixing screws are tightened to fix the sealing ring and the annular cover in a sealing manner.

### Test Procedure:

At the beginning, the filled and closed testing cup is weighed (weight A). After 24 hours in the conditioning cabinet, the testing cup is weighed again (weight B). It is important when filling and transporting to ensure that the upper rim of the cup and the test sample do not become wetted with the water.

### Evaluation:

The water vapor permeability, abbreviated as WVTR, is determined in the unit [g/m²/24 h] on the basis of the following equation:

$$W V T R [ g \text{/} {m}^{2} \text{/} 24 h ] = \frac{( A - B )}{test area [ {m}^{2} ]}$$

The test area corresponds to the opening of the steel testing cup.

### As a Modification of the Method Described Above:

In the case of investigations in which no test sample adapted to the test area of the circular sample holder is available or can be provided, such as for example in the case of relatively small and/or non-circular test samples, a test sample obtained by means of combining with a backing material may be used. For investigating the water vapor permeability, this test sample is produced by analogy with the test sample described above for investigating the air permeability. In such cases, when carrying out the measurements for water vapor permeability, corresponding correcting and standardizing measurements should also be carried out analogously at the same time and correspondingly taken into consideration in the evaluation.

In a development of the concept of the invention, it proves to be advantageous that the second joining region is in this case both arranged within the first region of the overhang and thereby respectively extends over a subregion of the overhang, and then additionally over the longitudinal periphery of the crotch part goes over into the subregion adjacent thereto of the stomach portion and/or of the back portion, therefore it is in this way introduced into the incontinence article as a region bridging the respective longitudinal periphery of the crotch portion.

The second joining region is consequently provided in a region bridging the respective longitudinal periphery of the crotch region, therefore both a subregion of the overhang and a subregion adjacent thereto of the stomach portion and/or of the back portion being extended over. The fact that the second joining region is provided in a region bridging the longitudinal periphery of the crotch portion means that the direct longitudinal periphery of the crotch portion is securely attached to the chassis materials of the respective stomach or back portion. This is advantageous since uncontrolled protrusion of the overhang of the crotch portion, and consequently uncontrolled folding that may lead to hard locations, are prevented.

More advantageously, the second joining region extends over the subregion of the stomach portion and/or of the back portion that is adjacent the longitudinal periphery of the crotch portion in the transverse direction respectively with a width P″.

More advantageously, the second joining region extends over the subregion of the stomach portion and/or back portion that is adjacent the longitudinal periphery of the crotch portion in the transverse direction respectively with a width P″ of greater than 1 mm, preferably greater than 2 mm, more preferably greater than 5 mm, but preferably of less than 60 mm, preferably less than 50 mm, more preferably less than 40 mm, more preferably less than 30 mm, more preferably less than 20 mm.

Particularly advantageously, the proportion P″/H of the respective subregion of the stomach portion and/or back portion that is extended over by the second joining region in the transverse direction with a width P″ with respect to the respective overhang with a width H is at least 0.01, particularly at least 0.04, more particularly at least 0.07, more particularly at least 0.10, but preferably at most 0.90, more particularly at most 0.80, more particularly at most 0.70, more particularly at most 0.60, more particularly at most 0.50, more particularly at most 0.40.

Particularly advantageously, the proportion P″/N of the respective subregion of the stomach portion and/or back portion that is extended over by the second joining region in the transverse direction with a width P″ with respect to the respective side region of the stomach portion and/or back portion with a width N is at least 0.01, particularly at least 0.015, more particularly at least 0.020, but preferably at most 0.35, particularly at most 0.30, more particularly at most 0.25, more particularly at most 0.20, more particularly at most 0.15, more particularly at most 0.10. This proportion of the second joining region, and consequently the reinforcing region, in the respective side region of the stomach portion and/or back portion allows adequate reinforcement to be achieved, without these chassis materials and their elasticating means fixed therein being impaired in their flexibility and also the way in which they act over a large surface area.

Advantageously, the second joining region, and consequently the reinforcing region, has an overall width, that is to say the sum of the width P′ and the width P″, of 5-60 mm, particularly of 10-50 mm, more particularly of 10-40 mm, more particularly of 10-30 mm.

In an advantageous way, the second joining region with the overall width P is arranged along the longitudinal periphery of the crotch portion, so that the ratio of the width P′ to the width P″ is preferably between 1:4 and 4:1, more preferably between 1:3 and 3:1, more preferably between 1:2 and 2:1, particularly preferably 1:1.

In a further advantageous way, the second joining region is arranged parallel to the longitudinal direction with a constant overall width P. This allows the second joining region to be introduced into the incontinence article in a technically uncomplicated manner, and consequently also more quickly and at lower cost.

The joining means already described above, that is to say adhesive and/or non-adhesive joining means, may be used individually or in combination for the second joining region adjacent to the longitudinal periphery of the crotch part and extending over a subregion of the stomach portion and/or back portion.

It is particularly preferred if the second joining region is introduced into the incontinence article as a region bridging the respective longitudinal periphery of the crotch portion, in particular in such a way that the part of the second joining region that is extended over the subregion of the overhang and the part of the second joining region that is extended over the adjacent subregion of the stomach portion and/or back portion are provided with the same joining means (for example with respect to the distinction as to whether they are adhesive or non-adhesive), more particularly in the same arrangement (for example with respect to over the full surface area or not over the full surface area).

It is particularly preferred if the part of the second joining region that is extended over the adjacent subregion of the stomach portion and/or back portion has non-adhesive joining means, in particular in the form of welding locations, in particular ultrasonic welding locations, thermal welding locations and/or calender welding locations.

More particularly, the part of the second joining region that is extended over the adjacent subregion of the stomach portion and/or back portion is formed by non-adhesive joining means, in particular in the form of welding locations, in particular ultrasonic welding locations, thermal welding locations and/or calender welding locations. More particularly, the non-adhesive joining means in the part of the second joining region that is extended over the adjacent subregion of the stomach portion and/or back portion are provided not over the full surface area but rather in the form of a pattern, particularly in the form of a punctiform and/or strip-form and/or linear arrangement and/or some other pattern-like arrangement and/or in combinations thereof.

More particularly, also additionally provided in the part of the second joining region that is extended over the adjacent subregion of the stomach portion and/or back portion are openings extending through a buildup of plies present in this region in the Z direction. The openings may in this case be introduced by methods that are known as a matter of routine in the art, such as for example by means of cutting out, punching out or by welding operations passing through the buildup of plies.

In a particularly advantageous way, the openings which are arranged within the adjacent subregion of the stomach portion and/or back portion that is extended over by the second joining region are formed by the joining means introduced into this part of the second joining region, in particular in the form of welding locations, more particularly ultrasonic welding locations, thermal welding locations and/or calendar welding locations.

The chassis-forming materials of the stomach portion and/or back portion preferably comprise nonwoven materials, such as spunbonded nonwovens (S), meltblown nonwovens (M), SM nonwovens, SMS nonwovens, SMMS nonwovens, carded nonwovens or through-air bonded carded nonwovens. Particularly preferably, the chassis-forming material of the stomach portion and/or back portion comprises spunbonded nonwoven. The nonwoven materials used for the stomach portion and/or back portion advantageously have a basis weight of 10-30 g/m², more preferably of 15-25 g/m². Particularly preferably, the stomach portion and the back portion comprise a spunbonded nonwoven of polypropylene, particularly with a basis weight of 15-25 g/m².

For the forming of the crotch portion, a backsheet material or a topsheet material with low basis weights, to be specific of 10-40 g/m² and 5-20 g/m², respectively, is preferably used. This advantageously realizes the softness, adaptability and drape that are desired for the user of the incontinence article in these sensitive regions of the body.

The chassis-forming shell materials of the crotch portion are further advantageously formed: The backsheet material particularly comprises a sheeting, particularly of a basis weight of 10-40 g/m². In particular, the backsheet comprises a sheeting which is liquid-impermeable during use, but at the same time breathable, that is to say water-vapor-permeable, particularly microporous. The water vapor permeability of the backsheet is particularly at least 300 g/m²/24 h, more particularly at least 500 g/m²/24 h, more particularly at least 1000 g/m²/24 h, more particularly at least 1500 g/m²/24 h, more particularly at least 2000 g/m²/24 h, more particularly at least 2500 g/m²/24 h, more particularly at most 6000 g/m²/24 h, more particularly at most 5000 g/m²/24 h, more particularly at most 4000 g/m²/24 h measured on the basis of the method described in ASTM E 96-00.

The sheeting may advantageously also be provided with a nonwoven coating, which can impart a textile look to the outer side of the incontinence article that is facing away from the body. The nonwoven coating preferably consists of a nonwoven material, particularly a spunbonded nonwoven of polypropylene, particularly with a basis weight of 7-25 g/m², 10-20 g/m², particularly of 12-17 g/m².

The topsheet material preferably comprises nonwoven materials, such as spunbonded nonwovens (S), meltblown nonwovens (M), SM nonwovens, SMS nonwovens, SMMS nonwovens, carded nonwovens or through-air bonded carded nonwovens.

The topsheet material may in this case preferably be formed only from topsheet material. More preferably, the topsheet material may be a composite of topsheet material and barrier means. In a further advantageous form, the topsheet material is a composite of a liquid-permeable topsheet material with longitudinal peripheries and adjacent longitudinal peripheral regions and hydrophobic barrier means joined onto the longitudinal peripheries or longitudinal peripheral regions on both sides of the topsheet material at joining locations. This composite provides an incontinence article which has different requirement profiles region by region, to be specific a liquid absorption in the central region and a slowing of the lateral escape of liquid at the peripheral regions.

Corresponding to the functionality, advantageous materials given below are used: The topsheet material preferably comprises nonwoven materials, such as spunbonded nonwovens, carded nonwovens or through-air bonded carded nonwovens. Particularly preferably, the topsheet material comprises spunbonded nonwoven. More advantageously, the nonwoven materials used for the topsheet have a basis weight of 5-20 g/m², 8-20 g/m², more preferably of 10-18 g/m², particularly preferably of 12-16 g/m². Particularly preferably, the topsheet comprises a hydrophilicized spunbonded nonwoven, particularly of polypropylene, particularly with a basis weight of 12-16 g/m².

The material of the barrier means preferably comprises nonwoven materials, such as spunbonded nonwovens, meltblown nonwovens, carded nonwovens or through-air bonded carded nonwovens. Particularly preferably, the material of the barrier means comprises single-ply or multi-ply nonwovens. Particularly preferably, the material of the barrier means comprises laminates of one or more plies of spunbonded nonwoven (S) and/or meltblown nonwoven (M), particularly SMS laminates or SMMS laminates, particularly based on polyolefins, such as for example polyethylene or polypropylene. Such materials are inexpensive and, on account of their inherently hydrophobic property, suitable for having a liquid-retardant effect.

More advantageously, the nonwoven materials used for the barrier means have a basis weight of 5-20 g/m², preferably of 8-20 g/m², more preferably of 10-18 g/m². Particularly preferably, the barrier means comprises a laminate of spunbonded nonwoven and meltblown nonwoven plies, particularly of polypropylene, particularly with a basis weight of 10-18 g/m².

In a further configuration, the hydrophobic barrier means extends over the longitudinal peripheries of the topsheet material, to be precise to form a barrier means respectively running in the longitudinal direction on both sides of the absorbent body that is in each case upright and is typically referred to as a cuff element or leg-band element. The distal ends of the barrier means are advantageously provided with elasticating means. In this way, the barrier means are raised against the surface of the user's skin during use of the incontinence article.

The fixing of the material webs of the topsheet-material composite at the joining locations may preferably take place by means of adhesive, particularly hotmelt adhesive, thermal calendering (thermobonding) or ultrasonic welding. The fixing may take the form of continuous joining locations, in order to achieve a high bonding force between the topsheet material and the barrier means. A continuous line is conceivable here. However, fixing by intermittently applied joining locations is also conceivable and advantageous, that is to say by a sequence of discrete points of attachment or lines of attachment or any other pattern of attachment.

The backsheet material and the topsheet material advantageously have the same extent in the transverse direction. They are congruent in relation to one another.

It is also more advantageous, however, if the backsheet material and the topsheet material are not congruent in relation to one another. Particularly advantageously, the backsheet material has a narrower extent in the transverse direction in comparison with the topsheet material. In this way, the backsheet material, such as for example a sheeting, which possibly detracts from the wearing comfort for the user, is covered over by the skin-friendly nonwoven material of the topsheet material.

In a development of the invention, it proves to be advantageous if the proportion of the overall surface area of the incontinence article that is made up by the surface area of the crotch portion is 25-55%, particularly 30-47%, more particularly 35-47% and more particularly 35-45%.

In a development of the invention, the overlapping region between the crotch portion and the stomach portion is formed such that the crotch portion overlaps 15-40%, particularly 15-35% and more particularly 15-25% of the surface area of the stomach portion. In an advantageous way, the crotch portion overlaps the stomach portion with a surface area of 25 000-45 000 mm².

In a development of the invention, the overlapping region of the crotch portion and the back portion is formed such that the crotch portion overlaps 20-40%, particularly 20-35% and more particularly 22-32% of the surface area of the back portion.

In an advantageous way, the crotch portion overlaps the back portion with a surface area of 35 000-65 000 mm², particularly of 40 000-55 000 mm².

The overlapping of the crotch portion with the back portion is advantageously greater than the overlapping of the crotch portion with the stomach portion.

In the case of the form of the incontinence article according to the invention, it is possible and advantageous if the absorbent body also overlaps 5-20%, particularly 5-15%, of the surface area of the stomach portion and/or 10-20%, particularly 10-15%, of the surface area of the back portion.

The extent of the stomach portion and of the back portion in the side seam region in the longitudinal direction is advantageously at least 100 mm, particularly at least 150 mm and particularly 150 mm to 220 mm.

The minimum distance between the stomach portion and the back portion in the longitudinal direction is advantageously 250 to 400 mm.

The maximum extent of the crotch portion in the transverse direction, that is to say the greatest width E, is advantageously at least 200 mm, particularly 200 to 350 mm, more particularly 250 to 320 mm.

Furthermore, it proves to be advantageous if the overhang of the backsheet material and/or of the topsheet material in the transverse direction is in total, that is to say on both sides of the longitudinal peripheries of the absorbent body, particularly 25-50%, more particularly 30-45% and more particularly 35-45%, with respect to the greatest width E of the crotch portion. The relatively large overhang of backsheet material and/or topsheet material on both sides of the absorbent body therefore means a wide crotch portion with a relatively narrow absorbent body. This makes it possible to provide in the crotch portion leg elasticating means that are made to extend along the leg openings and are at a relatively great distance from the bulky, and therefore rigid, absorbent body. This in turn results in good sealability and adaptability of the leg opening peripheries on both sides of the crotch portion. This is because the bulky absorbent body that is torsionally rigid in comparison with thin chassis materials is in this way of only little hindrance to the forming of a liquid-tight leg termination; it is therefore not necessary to work with extremely high tensions to form a liquid-tight leg termination, which in turn has a positive effect on the wearing comfort of the incontinence article.

In yet a further form of the invention, it proves to be particularly advantageous if the leg elasticating means end in the longitudinal direction at least 10 mm, particularly at least 20 mm, before the second elasticating means. It is particularly advantageous if the leg elasticating means end in the longitudinal direction before the stomach portion and/or before the back portion. The tension and restoring force exerted by them therefore does not influence the tension conditions of the second elasticating means. The tension conditions are particularly not influenced within the crotch-side region of the stomach portion and of the back portion that is facing the leg openings, in which the second elasticating means are provided in a fanning-out manner.

Preferably used as leg elasticating means are elasticating means in the form of threads or bands, such as rubber or polyether-polyurethane or polyester-polyurethane threads, preferably elastic threads such as Lycra®, Creora® or Spandex® threads. The leg elasticating means preferably have a thickness of 300-1500 dtex, particularly of 500-1200 dtex, more particularly 500-900 dtex. The leg elasticating means are preferably fixed with a pretensioning of 1.5-6.0, particularly of 2.5-4.5 on the chassis-forming shell materials of the crotch portion. The pretensioning is defined as a factor of the degree of stretching with respect to the unstretched/relaxed state of the elasticating means.

For the areal elastification of the stomach portion and back portion, the first elasticating means are provided, respectively spaced apart from one another and made to extend parallel to one another in the transverse or waist-encircling direction. These preferably have the same pretensioning and serve substantially for an areally continuous, uniform elastification of the stomach portion and of the back portion in the region well above the leg openings. It is however possible for the first elasticating means to have a stronger pretensioning in an upper peripheral region of the waist or for a number of these elasticating means to be provided in a closely spaced manner, in order to realize a somewhat stronger elastification at the periphery of the waist.

In yet a further form of the present invention, it has also been recognized that the tension conditions in said crotch-side region of the stomach portion and of the back portion that is facing the leg openings are essential with regard to the wearing comfort and can be made such that the wearing comfort is improved. Advantageously, the second elasticating means extend from the two side seam regions in the direction of a longitudinal center axis of the incontinence article and thereby run in an arcuately fanning-out manner with increasing distance from one another.

For this, the crotch-side region that is facing the leg openings, in which the second elasticating means fan out in the direction of the longitudinal center axis, is preferably formed such that, when this region is stretched over its surface area, the restoring force thereby occurring decreases in the direction of the crotch portion.

If this crotch-side region of the stomach portion and of the back portion that is facing the leg openings is thus considered, to be precise in a direction from the respective side seam region toward the crotch portion, that is to say in the direction of a longitudinal center axis of the incontinence article and to a certain extent in the direction of the arcuate fanning-out of the second elasticating means, the restoring force occurring in this direction when it is stretched out over its surface area is reduced. The force concerned here is therefore the force with which the stomach portion and the back portion resist stretching over the surface area. A decrease in this restoring force, which then of course is transferred to the user, is accompanied by a considerable improvement in the wearing comfort of the incontinence article.

It also proves to be particularly advantageous if the decrease in the restoring force in said crotch-side region of the stomach portion and of the back portion that is facing the leg openings is provided such that, in the direction of the crotch portion, a decreasing number of folds per centimeter are formed in the transverse direction of the incontinence article. In such a way, the stomach portion and the back portion can stretch in a way corresponding to the body shape of the user, without the elastic forces formed thereby causing the chassis material to gather with a multitude of folds. It should once again be explained at this point that the decrease in the restoring force in the direction of the crotch portion means that the force that is produced as a result of stretching over the surface area becomes less with increasing proximity to the crotch portion. The restoring force as a result of stretching over the surface area is therefore greater in an area nearer the side seam than in an area nearer the crotch portion.

Said tension conditions can be achieved in a variety of ways, for instance by using materials of different elasticity in the transverse direction in the crotch-side region that is facing the leg openings, in which the second elasticating means are also provided. It would also be conceivable for the pretensioning of the second elasticating means to be reduced with increasing proximity to the crotch portion, that is to say from the outside inward in the direction of a longitudinal center axis. It would also be conceivable for the decrease in the restoring force when stretching over the surface area to be achieved by increasing the distance between the second elasticating means, it having to be ensured here that this is not compensated by a strong increase in the pretensioning as a result of the fan-shaped line followed by the elasticating means, or even exceeded in the direction of increasing restoring force.

To determine the restoring forces, the regions of the chassis to be measured may be firmly clamped directly, as it were non-destructively, between two clamping jaws of a defined, identical clamping jaw width, and the restoring forces determined under defined stretching of the regions to be measured that simulates the state of use, by particularly 30% or 50% or 80% of the initial length (of the clamping jaw spacing when fixing the region to be measured in the unclamped state). The clamping jaws should fix as many elasticating means as possible, but at least two arranged next to one another, of the region to be measured and be oriented substantially perpendicularly with respect to the line followed by the elasticating means, so that the stretching takes place between the clamps substantially in the direction of the line followed by the elasticating means.

It has particularly proven to be advantageous if a minimum distance between the second elasticating means (spacing of elastificating means lying directly next to one another) in the side seam regions is 3 to 8 mm, particularly 3 to 7 mm and more particularly 3 to 6 mm.

Furthermore, it has proven to be advantageous if a maximum distance between the second elasticating means (spacing of elastificating means lying directly next to one another) at a periphery of the absorbent body or at a longitudinal periphery of the crotch portion is 7 to 35 mm, particularly 10 to 32 mm and more particularly 12 to 30 mm.

Furthermore, it has proven to be advantageous if the second elasticating means have a degree of fanning out F

F=(A−B)/B*100%

of 50 to 900%, particularly of 100 to 700% and more particularly of 150 to 550%.

The degree of fanning out F is defined as the ratio of the decrease in distance (A−B) to the minimum distance (B) in percent. The variables A and B are defined here as the distance of the outermost second elasticating means in the longitudinal direction from the innermost second elasticating means in the longitudinal direction (that is to say not the spacing of second elasticating means lying directly next to one another), to be precise A as the maximum distance, particularly at the longitudinal periphery of the crotch portion or at the periphery of the absorbent body, and B as the minimum distance particularly in the side seam region.

It has also been recognized that it proves to be advantageous if the degree of fanning out F of the second elasticating means is greater in the back portion than in the stomach portion.

On account of the natural shapes of the body in the back region or stomach region of a user, the problems addressed here typically prove to be more serious in the back or buttocks region. To this extent, it proves to be advantageous if the maximum distance between the second elasticating means at a periphery of the absorbent body is greater in the back portion than in the stomach portion.

In an advantageous embodiment, the respective second joining regions, and consequently the reinforcing regions, extend, when considered in the longitudinal direction, from the crotch-facing transverse periphery of the stomach portion and of the back portion in the direction of the longitudinal ends of the crotch portion, to be precise at least up to the length consequently covered by the second elasticating means running arcuately from the side seam regions in the direction of the longitudinal center axis of the incontinence article, and particularly fanning out with increasing distance and finishing at the longitudinal periphery of the crotch portion. More advantageously, the respective second joining regions, and consequently the reinforcing regions, extend from the crotch-facing transverse periphery of the stomach portion and of the back portion, when considered in the longitudinal direction, at least up to the length that coincides with the placement of an imaginary horizontal line level with the height of the side seam region from which the arcuately running, and particularly fanning-out, second elasticating means begin. As described at the beginning, the stiffening by means of the reinforcing region provided by the second joining region has an advantageous effect on the fit.

It would be entirely conceivable for the second elasticating means to run continuously from one side seam region to the other side seam region, which particularly simplifies introduction in a continuous production process in comparison with a “cut-and-place” process. As a result of the coverage of the crotch portion with the stomach portion and with the back portion, there may, depending on the design, also be an overlapping or coverage of the bulky absorbent body with the stomach portion and/or the back portion, and consequently also with that crotch-side region of the stomach portion and of the back portion that is facing the leg openings in which the second elasticating means run. The bulky absorbent body in this case usually hinders elastic stretchability of the chassis materials. Furthermore, it is not necessarily advantageous if the bulky absorbent body is subjected to additional tensioning forces. It may therefore prove to be advantageous if the second elasticating means are deactivated with regard to their elastic properties in an overlapping region with the absorbent body of the crotch portion. This deactivation may be realized, for example, by a number of separating cuts through the second elasticating means in the region of the coverage with the absorbent body, while other separating methods, such as for example by means of ultrasonic welding or laser, are also conceivable.

It should be mentioned that the first elasticating means may also be deactivated with regard to their elastic properties in the region of coverage with the absorbent body.

With regard to the overall dimensions of the incontinence article, it proves to be advantageous if the distance (C) of the crotch-facing innermost second elasticating means of the stomach portion from the corresponding crotch-facing innermost second elasticating means of the back portion is 250 to 420 mm.

The distance of the innermost, crotch-facing second elasticating means from the peripheral contour, bounding the leg openings, of the crotch-side region of the stomach portion and of the back portion that is facing the leg openings is preferably 2-40 mm, more preferably 3-30 mm, particularly preferably 4-15 mm.

Preferably used as first and/or second elasticating means are elasticating means in the form of threads or bands, such as rubber or polyether-polyurethane or polyester-polyurethane threads, preferably elastic threads such as Lycra®, Creora® or Spandex® threads. The first and/or second elasticating means preferably have a thickness of 300-1500 dtex, particularly of 500-900 dtex, more particularly 500-600 dtex. The first and/or second elasticating means are preferably fixed with a pretensioning of 1.5-6.0, particularly of 2.5-5.0, on the chassis-forming shell materials of the stomach portion and back portion. The pretensioning is defined as a factor of the degree of stretching with respect to the unstretched/relaxed state of the elasticating means.

Quite apart from this, it proves to be advantageous if, at least outside the absorbent body, the stomach portion and the back portion are transversely elasticated over the surface area substantially continuously over the longitudinal direction, it also being possible in this way to maintain or realize the advantageous tension conditions.

The absorbent body comprises materials that absorb bodily fluids, such as natural or synthetic fibers, particularly cellulose fibers, preferably in the form of cellulose fluff. The absorbent core preferably also comprises superabsorbent materials (SAP), particularly based on surface-crosslinked, partially neutralized polyacrylates.

The crotch portion or the longitudinal peripheries of the crotch portion which bound the leg openings are advantageously formed in an arcuately contoured manner.

Further features, details and advantages of the invention are provided by the accompanying patent claims and by the graphic representation and description that follows of a preferred embodiment of the incontinence article according to the invention. In the drawing:

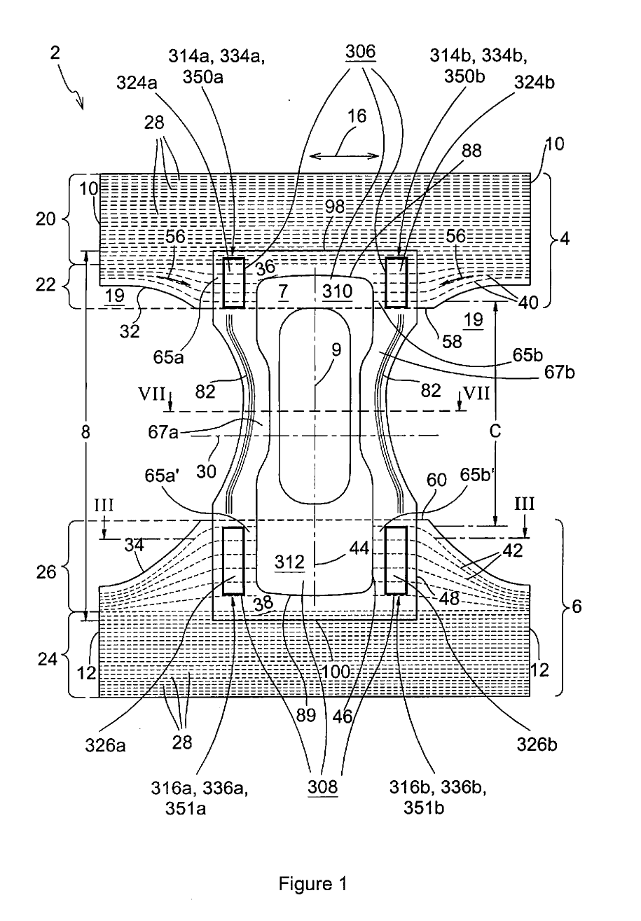

FIG. 1 shows a plan view of an incontinence article according to the invention, wherein a stomach portion, a back portion and a crotch portion connecting the two of the incontinence article have not yet been put together in the form of briefs, but are represented in the laid-flat and stretched-out state;

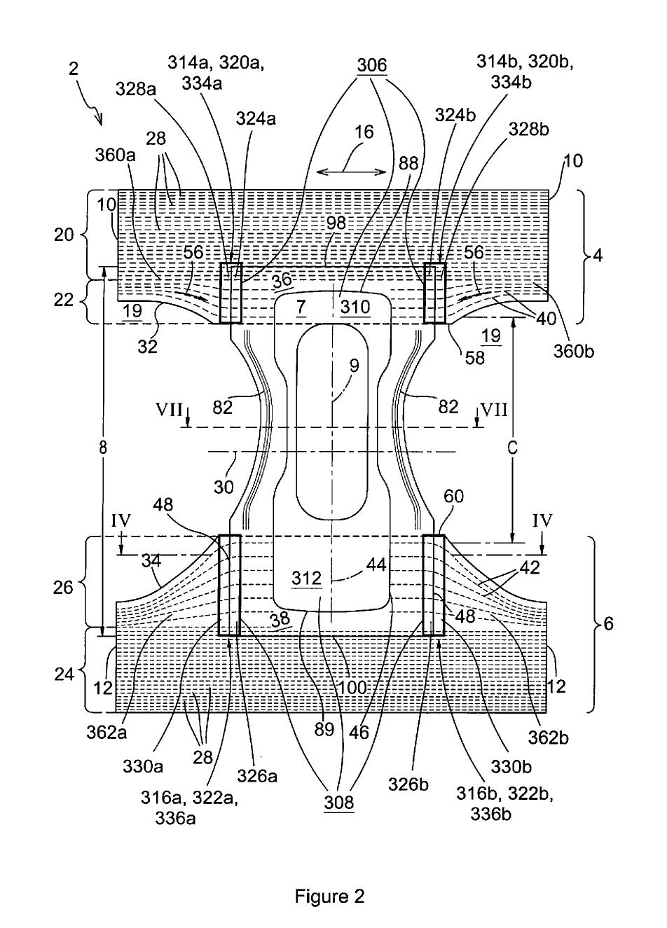

FIG. 2 shows a plan view of an incontinence article according to the invention, wherein a stomach portion, a back portion and a crotch portion connecting the two of the incontinence article have not yet been put together in the form of briefs;

FIG. 3 shows a sectional view (schematically) along a transverse center axis of the crotch portion and back portion attached thereto with the sectional plane III-III in FIG. 1;

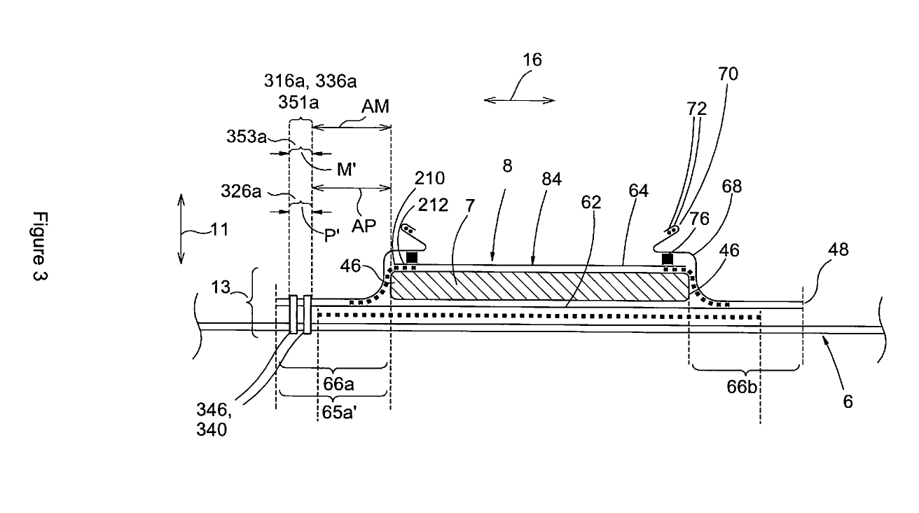

FIG. 4 shows a sectional view (schematically) along a transverse center axis of the crotch portion and back portion attached thereto with the sectional plane IV-IV in FIG. 2;

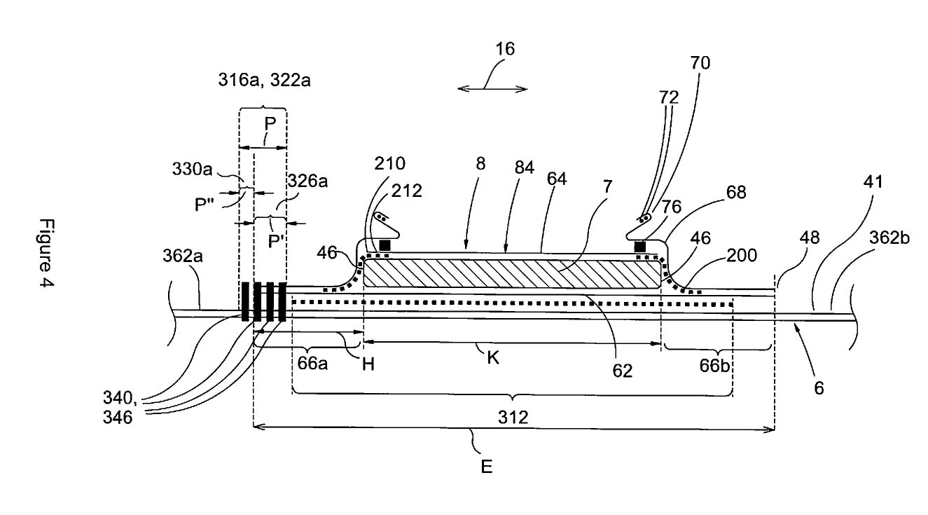

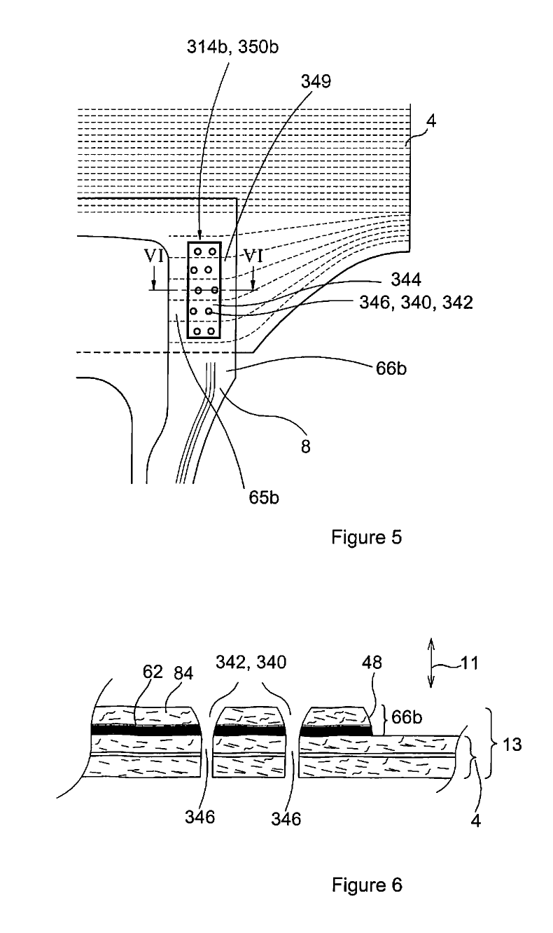

FIG. 5 shows a representation of a second joining region and an air-permeable region of the incontinence article that is shown in FIG. 1 in the form of a detail;

FIG. 6 shows a sectional view (schematically) of the second joining region and the air-permeable region with the sectional plane VI-VI in FIG. 5;

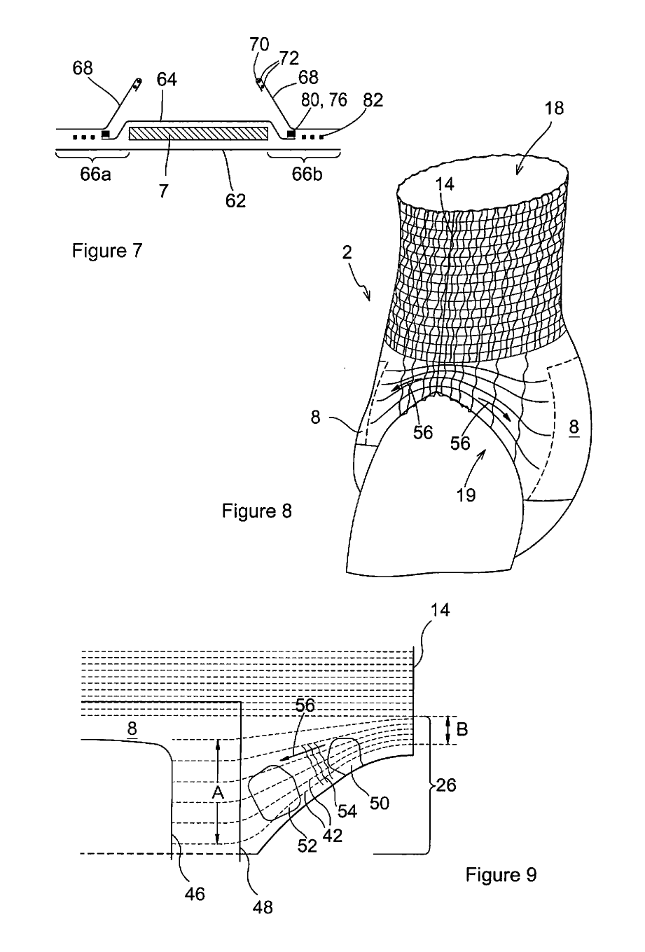

FIG. 7 shows a sectional view (schematically) along a transverse center axis of the crotch portion with the sectional plane VII-VII in FIG. 1;

FIG. 8 shows a perspective view (schematically) of the incontinence article that is shown in FIG. 1 or 2 put onto a user;

FIG. 9 shows a representation of the incontinence article that is shown in FIG. 1 or 2 in the form of a detail;

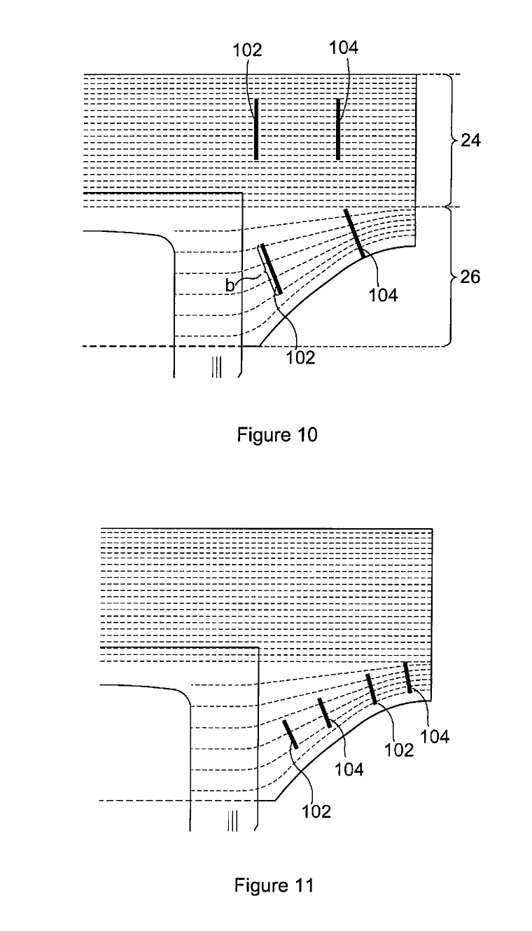

FIGS. 10, 11 illustrate by way of example the determination of restoring forces in the stomach portion and back portion of the incontinence article;

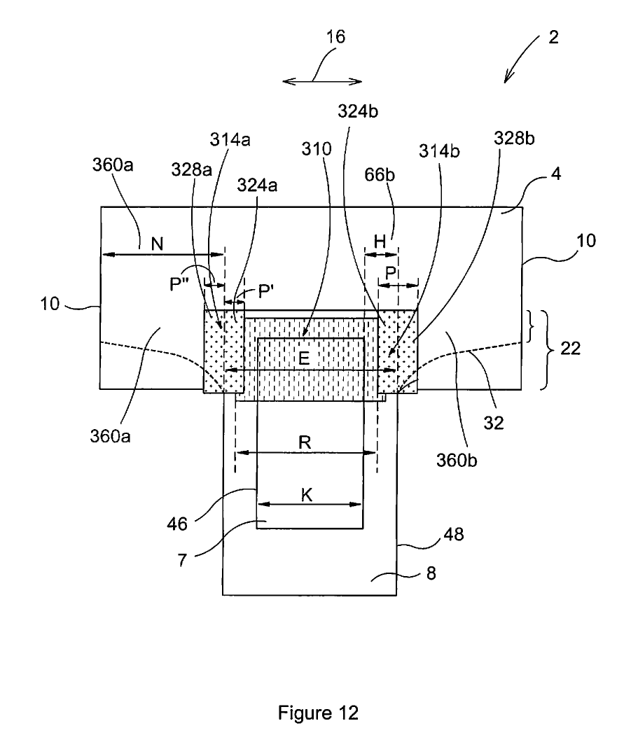

FIG. 12 shows a plan view of a schematically represented incontinence article in the laid-flat and stretched-out state, wherein the crotch portion is shown initially only connected to a stomach portion.

The figures show an incontinence article in the form of briefs, designated overall by the reference numeral 2, for receiving solid and liquid body excretions. The incontinence article 2 is formed by three components that can, to the greatest extent, be produced independently of one another, to be specific a front stomach portion 4, a rear back portion 6 and a crotch portion 8, which is arranged between said stomach portion and said back portion and has an absorbent body 7, wherein the crotch portion 8 overlaps with a substantial proportion of the surface area the stomach portion 4 on the one hand and the back portion 6 on the other hand and in the overlapping region is inseparably connected at the manufacturer's.

As can be seen from FIGS. 1 and 2, this leads to an H-shaped basic structure of the incontinence article with a longitudinal direction 9. To form the briefs form that is schematically represented in FIG. 8, the joined-together component parts represented in FIGS. 1 and 2 are then connected to one another, likewise at the manufacturer's, at respective lateral longitudinal peripheral portions 10, 12 of the stomach portion 4 and of the back portion 6, whereby side seam regions 14 (FIG. 8) are formed on both sides. In this state of the incontinence article in the form of briefs that is produced at the manufacturer's, the stomach portion 4 and the back portion 6 extend in the transverse or waist-encircling direction 16 continuously up to the side seam regions 14 and thus define a waist opening 18, which is closed in the waist-encircling direction, and leg openings 19, through which the user puts on the incontinence article like a pair of briefs.

The stomach portion 4 can be subdivided into a waist-side region 20 and a crotch-side region 22 that is facing the leg openings. A corresponding subdivision is provided in the back portion 6, to be precise likewise into a waist-side region 24 and a crotch-side region 26 that is facing the leg openings.

Provided in the waist-side region 20 of the stomach portion 4 and in the waist-side region 24 of the back portion 6 are first elasticating means 28, which may particularly be elasticating means in the form of threads, such as Lycra® threads, which in the pre-stretched state are connected to the flat materials (chassis materials) of the stomach portion 4 and of the back portion 6 in what is known as the stretch-bonding process. These first elasticating means 28 extend in the transverse or waist-encircling direction 16 from one side seam region 14 to the other.

The crotch-side portion 22 of the stomach portion 4 or crotch-side portion 26 of the back portion 6 that is facing the leg openings 19 have a peripheral contour 32 or 34 deviating from the transverse or waist-encircling direction 16 and running in the direction of a transverse center axis 30 of the crotch portion 8. This peripheral contour 32, 34 is also arcuate in the representation according to FIG. 1 or 2 and therefore suitable for bounding the leg openings 19. This shape of the crotch-side region 22 or 26 that is facing the leg openings also creates a relatively great overlapping region 36, 38 between the crotch portion 8 and the stomach portion 4 or the back portion 6, which is essential with regard to a tear-resistant connection of the crotch portion 8 and the stomach portion 4 or the back portion 6.

The respective crotch-side region 22, 26 that is facing the leg openings 19 of the stomach portion 4 and of the back portion 6 is likewise of an elasticated form. Respectively provided there are second elasticating means 40 and 42. The second elasticating means 40, 42 in each case extend from the side seam regions 14 in the direction of a longitudinal center axis 44 of the incontinence article. As can be seen from FIGS. 1 and 7, the second elasticating means 40, 42 fan out in the direction of the longitudinal center axis 44. This means that the spacing between them increases in the direction of the longitudinal center axis 44. This fanning out of the second elasticating means 40 and 42 can also be quantitatively specified more precisely on the basis of FIG. 9. For example, the second elasticating means 42 of the back portion 6 that are represented in FIG. 9 have in the side seam regions 14 a minimum distance from one another of 3 to 8 mm (spacing of elasticating means lying directly next to one another) and at a periphery 46 of the absorbent body or a longitudinal periphery 48 of the crotch portion 8 a maximum distance (spacing of elasticating means lying directly next to one another) from one another of 7 to 35 mm. On the basis of FIG. 9, a degree of fanning out F can also be defined as follows:

F=(A−B)/B*100%

This degree of fanning out may advantageously lie between 50 and 900%, particularly between 100 and 700% and more particularly between 150 and 550%. It is advantageously greater in the back portion 6 than in the stomach portion 4. The variables A and B are defined here as the distance of the outermost second elasticating means 40, 42 in the longitudinal direction 9 from the innermost second elasticating means 40, 42 in the longitudinal direction 9 (that is to say not the spacing of elasticating means lying directly next to one another), to be precise A as the maximum distance, particularly at the longitudinal periphery 48 of the crotch portion 8, and B as the minimum distance, particularly in the side seam region 14 (cf. FIG. 9).

If the degree of fanning out is chosen to be sufficient in the case of the second elasticating means 40, 42, a decreasing restoring force can in this way be realized within the crotch-side region 22 or 26 that is facing the leg openings 19 in the direction 56 of the crotch portion 8, as long as it is ensured that the arcuate shape of the second elasticating means 40, 42 that is facing away from the waist or transverse direction 16 does not cause an excessive increase in the pretensioning as a result of the greater path followed by these second elasticating means 40, 42. If an area 50 of the crotch-side region 22 or 26 concerned that is lying nearer the side seam region 14 is considered along with an area 52 that is lying nearer the crotch portion 8, the restoring force that occurs under stretching over the surface area of the area 52 (stretching in the direction of the elasticating means 42) is less than the restoring force that occurs under stretching of the area 50. This advantageously has the effect that, as a result of the lower elastic forces that are exerted by the second elasticating means 40, 42 in the case represented by way of example, the chassis materials of the stomach portion 4 and of the back portion 6 are less gathered, such that there are a smaller number of folds/crimps 54, to be precise in the direction of the crotch portion 8 from the respective side seam region 14. The fact that the restoring forces occurring when the stomach portion stretches over the surface area in the crotch-side region 22 of the stomach portion 4 or the crotch-side portion 26 of the back portion 6 facing the leg openings decrease in the direction of the arrow 56, that is to say generally in the direction of the crotch portion 8 from the side seam region 14, means that a considerable improvement in the wearing comfort is achieved because—as stated—elastically stretchable materials prove to be particularly problematic specifically in these regions, because these materials are particularly subjected to tension and stretching in these regions in accordance with the physiognomy of the form of the human body. A deliberately and advantageously provided reduction in this restoring force, that is to say decreasing restoring force in the direction of the arrow 56, that is in the direction of increasing proximity to the crotch portion 8, has the effect here of providing a hitherto unachieved degree of freedom.

As stated at the beginning, restoring forces may be determined directly on the chassis of the incontinence article. For this, the region concerned of the stomach portion 4 or of the back portion is clamped between two clamping jaws 102, 104 (see FIGS. 10, 11) of a defined, identical clamping jaw width (b), and restoring forces are then determined under defined stretching of the regions to be measured that simulates the state of use, by particularly 30% or 50% or 80% of the initial length (of the clamping jaw spacing in the unclamped state). The clamping jaws 102, 104 are in this case respectively moved away from one another. The clamping jaws 102, 104 should fix as many elasticating means 40, 42 as possible, but at least two arranged next to one another, of the region to be measured and they should be oriented substantially perpendicularly with respect to the line followed by the elasticating means, so that the stretching between the clamping jaws 102, 104, that is to say the moving apart of the clamping jaws 102, 104, takes place substantially in the direction of the line followed by the elasticating means. This is realized in FIGS. 10 and 11.

In the case of the preferred embodiment of the incontinence article 2 that is represented, a distance C of the crotch-facing innermost second elasticating means 40 of the stomach portion 4 from the corresponding crotch-facing innermost second elasticating means 42 of the back portion 6 is between 250 and 420 mm, depending on the manufactured size of the incontinence article. The second elasticating means 40, 42 extend substantially up to the crotch-facing transverse periphery 58, 60 of the stomach portion 4 and of the back portion 6. The spacing of stomach portion 4 and back portion 6 from one another is 250-400 mm.

The distance of the innermost, crotch-facing second elasticating means 40, 42 from the peripheral contour 32, 34, bounding the leg openings, of the crotch-side region 22, 26 of the stomach portion 4 and of the back portion 6 that is facing the leg openings is preferably 2-40 mm, more preferably 3-30 mm, particularly preferably 4-15 mm.

The extent of the stomach portion 4 and of the back portion 6 in the side seam region 14 in the longitudinal direction 9 is advantageously between 100 and 220 mm. The extent of the crotch portion 8 in the transverse direction 16 is advantageously 200 to 350 mm.

As can be seen in FIGS. 3 and 4, which schematically represent a sectional view respectively along the sectional plane II-II from FIG. 1 and along the sectional plane IV-IV from FIG. 2, the crotch portion 8 comprises a liquid-impermeable backsheet material 62, which may be formed particularly by a breathable, but liquid-impermeable sheeting material, and a topsheet material 84, preferably produced on a nonwoven basis, which is a composite of a topsheet material 64 produced on a nonwoven basis and barrier means 68 arranged on both sides. As can be seen from FIGS. 3 and 4, arranged between the backsheet material and the topsheet material is the absorbent body 7. The absorbent body 7 has longitudinal peripheries 46. In the case represented by way of example, the backsheet material 62 respectively forms an overhang 66a, 66b in the transverse direction 16 on both sides of the longitudinal peripheries 46. The topsheet 64 protrudes only relatively slightly beyond the absorbent body 7 in the transverse direction; however, an upright barrier means 68 is respectively provided, running on both sides of the absorbent body 7 in the longitudinal direction 9, typically referred to as an upright cuff element or leg-band element and preferably formed by a hydrophobic, particularly liquid-impermeable nonwoven material, which extends in the transverse direction 16 preferably up to the lateral longitudinal peripheries 48 of the crotch portion 8. The barrier means 68 is in this case joined onto the longitudinal peripheries 210 or the longitudinal peripheral regions 212 of the topsheet material 64 at joining locations 76. The distal ends 70 of the barrier means 68 are provided with further elasticating means 72, which raise the barrier means 68 against the surface of the user's skin during use of the incontinence article, as is represented in FIG. 7, a schematic sectional view along the sectional plane VII-VII from FIG. 1 or FIG. 2. The joining locations 76 arranged in the longitudinal direction 9 form the continuously extended cuff base line 80.

The mentioned overhang 66a, 66b of the backsheet material 62 and/or of the topsheet material 84 on both sides of the longitudinal peripheries 46 of the absorbent body 7, that is to say in total, is at least 25% with respect to the greatest width E of the crotch portion 8. A great overhang of the backsheet material or of the topsheet material and backsheet material in the transverse direction respectively outside the longitudinal peripheries of the absorbent body has the effect that, on account of the accompanying great overlapping region of the crotch portion and the stomach portion and of the crotch portion and the back portion, the crotch portion can be securely fixed to the back portion and the stomach portion at the manufacturers.

A great overhang is additionally advantageous because in this way there is space in the transverse direction 16 for the arrangement of leg elasticating means 82 to extend along the leg openings 19. This is because it proves to be advantageous if the leg elasticating means 82 run at a certain distance from the bulky, and consequently rather rigid, absorbent body 7, in order on the one hand not to exert any additional stretching or torsional forces on the absorbent body, which could adversely influence its absorption behavior, and on the other hand to realize a liquid-tight leg termination that is largely uninfluenced by the absorbent body. It proves to be particularly advantageous in the case represented that these leg elasticating means 82 end in the longitudinal direction 9 at a clear distance of particularly at least 10 mm, preferably at least 20 mm, before the second elasticating means 40 and 42 of the stomach portion 4 or of the back portion 6. These leg elasticating means 82 preferably end ahead of the stomach portion 4 and the back portion 6 in the longitudinal direction 9. This is advantageous and essential because the leg elasticating means 82 in this way have little or no influence on the stress behavior of the stomach portion 4 and the back portion 6. This is because it has been recognized that it proves to have an adverse effect on the aim to be advantageously achieved of improving the wearing comfort precisely in the crotch-side region 22 and 26 of the stomach portion 4 and of the back portion 6 that is facing the leg openings 19 if the leg elasticating means 82, which are usually formed with great pretensioning and a correspondingly great restoring force, additionally run there.

As can be seen from FIG. 3 or 4 in conjunction with FIG. 1 or 2, in the case of the crotch portion 8 a relatively great overhang 66a, 66b in the transverse direction 16 is realized on both sides of the longitudinal peripheries 46 of the absorbent body 7, to be precise in particular also at regions of the crotch portion 8 that are facing the stomach portion 4 and the back portion 6, respectively. As a result—as already pointed out—a relatively great overlapping region 36, 38 of the crotch portion 8 with the stomach portion 4 and with the back portion 6 is realized. According to a preferred embodiment variant, the overlapping region 36 of the crotch portion 8 with the stomach portion 4 comprises at least 12% of the surface area of the stomach portion 4, and the overlapping region 38 of the crotch portion 8 with the back portion 6 comprises at least 20% of the surface area of the back portion 6. This proves to be advantageous since in this way a secure fixing of the crotch portion 8 on the stomach portion 4 and on the back portion 6 can be achieved.

The overhang 66a, 66b has in this case “first regions” 65a, 65a′, 65b, 65b′ and “second regions” 67a, 67b (see FIG. 1), a first region respectively extending on both sides of the longitudinal peripheries 46 of the absorbent body 7 in the front and rear overlapping regions 36, 38. A respective second region 67a, 67b extends on both sides of the longitudinal peripheries of the absorbent body between the transverse peripheries 58, 60 of the stomach portion 4 and of the back portion 6 that are facing the crotch, that is to say outside the overlapping regions.

As can be seen in FIGS. 1 and 2, the incontinence article 2 has the first joining regions 310, 312 and second joining regions 314a, 314b, 316a, 316b according to the invention, which are arranged in the front and rear overlapping regions 36, 38, and consequently provide a front and a rear connecting region 306, 308, in which the crotch portion 8 is inseparably joined onto the stomach portion 4 and the back portion 6, respectively.

FIG. 1 also schematically shows the second joining regions 314a, 314b, 316a, 316b, which are arranged at least within the first regions 65a, 65a′, 65b, 65b′ of the overhang 66a, 66b of the crotch portion 8, such that the second joining regions 314a, 314b, 316a, 316b form reinforcing regions 334a, 334b, 336a, 336b. Considered together with FIG. 3, which represents a schematic sectional view along the plane III-III from FIG. 1 (although only the second joining region on the left side is schematically depicted here), also explained are the air-permeable regions 350a, 350b, 351a, 352b, which in the exemplary embodiment shown coincide particularly advantageously in their areal extent with the respective reinforcing regions 334a, 334b, 336a, 336b.

In the overlapping regions 36, 38 there is a buildup of plies 13 in the Z direction 11, the Z direction extending perpendicularly to the plane described by the longitudinal direction 9 and transverse direction 16. Thus, a buildup of plies 13 is formed for example by overlapping of the crotch portion 8 with the back portion 6 (see FIG. 3).

It can be seen from FIG. 3 that the first region 65a′ of the overhang 66a, arranged in the rear overlapping region, has openings 346 extending through the buildup of plies 13 in the Z direction 11. The second joining region 316a thereby extends over a subregion 326a of the overhang 66a in the transverse direction 16 with a width P′. The air-permeable region 351a formed by the openings 346 extends over a subregion 353a of the overhang 66a in the transverse direction 16 with a width M′. In a particularly preferred embodiment, the openings 346 are formed by joining means 340 in the form of welding locations. The second joining region 316a represented forms a reinforcing region 336a and, by means of the openings 346 arranged within the second joining region, also represents the air-permeable region 35a. The two regions coincide in their areal extent and are mounted congruently one on top of the other. The second joining region 316a ends at a distance AP before the longitudinal periphery 46 of the absorbent body 7. Similarly, the air-permeable region 351a ends at a distance AM before the longitudinal periphery 46 of the absorbent body 7.

As also represented in FIG. 5 (here a detail of the incontinence article with the attachment of the crotch portion 8 to the stomach portion 4), the openings 346 within the first region 65b of the overhang 66b are arranged in a punctiform manner. The openings have been introduced by means of joining means 340 in the form of ultrasonic welding locations. The joining means respectively form a joining location (342), which is spaced apart from the next joining location by an unattached region 344. Correspondingly, the openings 346 are spaced apart from one another by a non-perforated region 344. To determine the areal extent of a second joining region (here 314b) and of the air-permeable region 350b, the respectively outermost joining means 340 or joining locations 342 or openings 346 are connected at their outermost peripheral edges by means of an imaginary line 349.

The openings 346 and the joining means 340 arranged in a point pattern form in the overall surface area that is extended over by the air-permeable region 350b and in the overall surface area that is extended over by the second joining region 314b openings or joining areas which, in total, assume preferably at least 1.5%, particularly at least 2.0%, more particularly at least 2.3%, more particularly at least 2.5% and preferably at most 20.0%, particularly at most 15.0%, more particularly at most 10.0%, more particularly at most 8.0%, more particularly at most 7.0%, more particularly at most 6.0%, with respect to the overall surface area extended over by the air-permeable region and by the second joining region, respectively. The individual openings or joining means have a diameter preferably of at least 0.2 mm, particularly of at least 0.3 mm, more particularly of at least 0.4 mm and preferably of at most 2.5 mm, particularly at most 2.0 mm, more particularly at most 1.5 mm, more particularly at most 1.2 mm, more particularly at most 1.0 mm. The neighboring individual openings 346 or joining means 340 present in the point pattern are spaced apart from one another by preferably 1-10 mm, particularly by 1-8 mm, more particularly by 1-6 mm, more particularly by 1-5 mm, more particularly by 1.5-4.5 mm, more particularly by 2-4 mm.

FIG. 6 represents a sectional view of the air-permeable region 350b or of the second joining region 314a along the sectional plane VI-VI from FIG. 5. The overhang 66b of the crotch portion 8, consisting of a topsheet material 84 of nonwoven and a backsheet 62 of sheeting, is attached to the stomach portion 4 lying thereunder by means of joining locations 342 produced by the joining means 340. Moreover, openings 346 extending through the buildup of plies 13 in the Z direction 11 have been introduced by the joining means 340.

Together with FIG. 4, which represents a schematic sectional view along the plane IV-IV from FIG. 2 (although only the second joining region on the left side is schematically depicted here), a further preferred embodiment is presented in more detail, to be specific that the second joining region is in this case arranged within the first region of the overhang and thereby respectively extends over a subregion of the overhang, and then additionally over the longitudinal periphery of the crotch part goes over into the subregion adjacent thereto of the stomach portion and/or of the back portion, therefore it is in this way introduced into the incontinence article as a region bridging the respective longitudinal periphery of the crotch portion.

It can be seen that the second joining region 314a, 314b, 316a, 316b is in this case arranged in a region 320a, 320b, 322a, 322b bridging the respective longitudinal periphery 48 of the crotch portion 8. Consequently, both a subregion 324a, 324b, 326a, 326b of the overhang 66a, 66b and a subregion 328a, 328b, 330a, 330b of the stomach portion 4 or back portion 6 that is adjacent the longitudinal periphery 48 is respectively extended over by the second joining region 314a, 314b, 316a, 316b with the joining means 340 arranged therein. These second joining regions 314a, 314b, 316a, 316b in each case form a reinforcing region 334a, 334b, 336a, 336b. The second joining regions 314a, 314b, 316a, 316b, and consequently the reinforcing regions 334a, 334b, 336a, 336b, extend, when considered in the longitudinal direction 9, from the crotch-facing transverse periphery 58, 60 of the stomach portion 4 and of the back portion 6 continuously in the direction of the longitudinal ends of the crotch portion, preferably to at least a respective longitudinal end 98, 100 of the crotch portion 8.

The second joining regions 314a, 314b, 316a, 316b have an overall width P of preferably 5-60 mm, particularly of 10-50 mm, particularly of 10-40 mm, more particularly 10-30 mm. In the subregion 324a, 324b, 326a, 326b of the respective overhang 66a, 66b that is extended over by the second joining region 314a, 314b, 316a, 316b, the second joining region has a width P′ of preferably greater than 1 mm, more preferably greater than 2 mm, more preferably greater than 5 mm but preferably less than 60 mm, more preferably less than 50 mm, more preferably less than 40 mm, more preferably less than 30 mm, more preferably less than 20 mm, particularly preferably of 10 mm.

The second joining region 314a, 314b, 316a, 316b extends over the respective subregion 324a, 324b, 326a, 326b of the overhang 66a, 66b in the transverse direction in such a way that the proportion P′/H of the subregion 324a, 324b, 326a, 326b of the respective overhang 66a, 66b that is extended over by the second joining region 314a, 314b, 316a, 316b, with the width P′ with respect to the respective overhang 66a, 66b, with the width H, is preferably at least 0.01, particularly at least 0.04, more particularly preferably at least 0.07, more particularly at least 0.10, but preferably at most 0.90, more particularly at most 0.80, more particularly at most 0.70, more particularly at most 0.60, more particularly at most 0.50, more particularly at most 0.40.

In the subregion 328a, 328b, 330a, 330b of the stomach portion 4 or back portion 6 that is extended over by the second joining region 314a, 314b, 316a, 316b, the second joining region has a width P″ preferably greater than 1 mm, more preferably greater than 2 mm, more preferably greater than 5 mm but preferably less than 60 mm, more preferably less than 50 mm, more preferably less than 40 mm, more preferably less than 30 mm, more preferably less than 20 mm, particularly preferably of 10 mm.

The second joining region 314a, 314b, 316a, 316b extends over the respective subregion 328a, 328b, 330a, 330b of the stomach portion 4 and of the back portion 6 in the transverse direction 16 in such a way that the proportion P″/H of the respective subregion 328a, 328b, 330a, 330b of the stomach portion and back portion that is extended over by the second joining region 314a, 314b, 316a, 316b, with the width P″ with respect to the respective overhang 66a, 66b with the width H, is preferably at least 0.01, particularly at least 0.04, more particularly at least 0.07, more particularly at least 0.10, but preferably at most 0.90, particularly at most 0.80, more particularly at most 0.70, more particularly at most 0.60, more particularly at most 0.50 and more particularly at most 0.40.

As explained above, the second joining region 314a, 314b, 316a, 316b extends over an adjacent subregion 328a, 328b, 330a, 330b of the stomach portion and of the back portion. Between the longitudinal periphery 48 of the crotch portion 8 and the longitudinal peripheral portion 10, 12 of the stomach portion 4 and back portion 6 are the respective side regions 360a, 360b, 362a, 362b. The respective side region has a width N. As also schematically represented in FIG. 12, the respective side region 360a, 360b (and 362a, 362b, respectively) of the stomach portion and of the back portion with the width N is extended over by the second joining region 314a, 314b (and 316a, 316b, respectively) with the proportionate width P″. In FIG. 12, the stomach portion or back portion is represented as a rectangular panel. However, as represented in FIG. 1 and also as depicted by dashed lines in FIG. 12, the region 22 facing the crotch may also assume an arcuate peripheral contour 32.

The proportion P″/N of the respective subregion 328a, 328b, 330a, 330b of the stomach portion and/or back portion that is extended over by the second joining region 314a, 314b, 316a, 316b, with the width P″ with respect to the respective side region 360a, 360b, 362a, 362b of the stomach portion and/or back portion with the width N, is preferably at least 0.01, particularly at least 0.015, more particularly at least 0.020, and preferably at most 0.35, particularly at most 0.30, more particularly at most 0.25, more particularly at most 0.20, more particularly at most 0.15, more particularly at most 0.10.

In an advantageous way, the proportion P′/N of the respective subregion 324a, 324b, 326a, 326b of the respective overhang 66a, 66b that is extended over by the second joining region 314a, 314b, 316a, 316b, with the width P′ with respect to the respective side region 360a, 360b, 362a, 362b of the stomach portion and/or back portion with the width N, is preferably at least 0.01, particularly at least 0.015, more particularly at least 0.020 and preferably at most 0.35, particularly at most 0.30, more particularly at most 0.25, more particularly at most 0.20, more particularly at most 0.15, more particularly at most 0.10.

The configuration of the incontinence article that is schematically represented in FIG. 4 together with FIG. 2 has second joining regions which are both arranged within the first region of the overhang and thereby respectively extend over a subregion of the overhang, and then additionally over the longitudinal periphery of the crotch part go over into the subregion adjacent thereto of the stomach portion and/or of the back portion. The openings 346 are formed at the same time by the joining means 340 introduced into the second joining region, in particular by means of joining means in the form of welding locations, preferably ultrasonic welding locations, thermal welding locations or calender welding locations.

As represented in FIG. 4, the first joining region 312 runs beneath the absorbent body 7 with the width K, therefore also extends over the contour, that is to say the respective longitudinal periphery 46 of the absorbent body 7, but not outside the contour of the crotch portion 8, but instead ends within the longitudinal peripheries 48 of the crotch portion 8. The crotch portion 8 is in this case preferably connected to the back portion 6 by means of an application of adhesive over the full surface area.

As in the present example, the topsheet material is a composite of a topsheet 64 and barrier means 68 joined on both sides onto the longitudinal peripheries 210 or the longitudinal peripheral regions 212 of the topsheet 64. The topsheet material is in this case attached by means of an adhesive 200, particularly a hotmelt adhesive, to the absorbent body and the side periphery 46 of the absorbent body 7, overlapping also an adjacent subregion of the backsheet material 62.

A separate joining means (not represented in FIG. 4) is also provided between the backsheet material and the absorbent body and between the topsheet material and the absorbent body. These separate joining means are applied in the form of an adhesive, not over the full surface area but in the form of an interrupted pattern. These separate joining means, provided in the form of an adhesive, are therefore applied for example in grid form, strip form or as a spiral pattern.

FIG. 12 schematically shows a not yet completed incontinence article 2 with a stomach portion 4 joined onto the crotch portion 8 and an advantageous configuration of the arrangement of the first joining region 310 and of the respectively second joining regions 314a, 314b. As represented in FIG. 12, the first joining region 310 extends in the transverse direction 16 with the width R advantageously over the longitudinal peripheries 46 of the absorbent body 7 with a width K, but without reaching over the longitudinal peripheries 48 of the crotch portion 8. The respectively second joining region 314a, 314b with the width P respectively bridges the longitudinal periphery 48 of the crotch portion 8. The joining region 314a, 314b in this case respectively extends over an adjacent subregion 324a 324b of the overhang 66a, 66b with the width H, to be precise with the width P′. The second joining region 314a, 314b also respectively extends over an adjacent subregion 328a, 328b of the stomach portion 4, to be precise with the width P″. The first joining region 310 thereby extends in the transverse direction in such a way that an overlap with the respective second joining region 324a, 324b is obtained.

An incontinence article in the form of briefs comprising the three components the crotch portion, the stomach portion and the back portion, wherein the crotch portion is joined inseparably to the stomach portion and back portion in overlapping regions while forming a buildup of plies in the Z direction, has according to the invention openings extending through this buildup of plies in the Z direction in the first region of the overhang. The openings form an air-permeable region, as explained above on the basis of the schematic representations of the figures. In the embodiment that is schematically represented in FIG. 5, the air-permeable region 350b having the openings 346 is arranged within the first region 65b, surrounded by a region without openings. The openings are arranged in a point pattern, the sum of the openings with a diameter of 0.5 mm assuming a proportion of 2.8% with respect to the overall surface area extended over by the air-permeable region. The air-permeable region has in this case an air permeability which is greater than the air permeability in the first region of the overhang outside the air-permeable region (350b) by a factor of 1.5, particularly 2, particularly 2.5, more particularly 3, more particularly at most 5, more particularly at most 4.5. In this exemplary embodiment, the air-permeable region has a width M′ of 20 mm, and, substantially beginning from the crotch-facing transverse periphery of the stomach portion 4, when considered in the longitudinal direction, extends in the direction of the longitudinal end of the crotch portion. A test piece with appropriately adapted dimensions, in the actual application example 10×50 mm, is punched out from the air-permeable region. The test piece punched out from this buildup of plies in this case comprises the plies introduced by the stomach portion and the crotch portion. In the exemplary embodiment, the test piece has two plies of nonwoven from the stomach portion, one ply of topsheet material in the form of an SMS laminate and the backsheet material as a nonwoven-sheeting laminate with a microporous sheeting. The investigation of the air permeability is performed on the basis of the measuring method described at the beginning. The test sample 1 necessary for measuring the air permeability for the test area of 20 cm² is produced by the test piece that has been punched out from the air-permeable region 350b being adhesively fixed, with overlapping on both sides, by means of an adhesive tape, such as for example crystal clear Tesafilm®, 15 mm×10 m from the company Tesa, in the punched-out region (7×45 mm) of a backing material, such as for example the air- and water-impermeable backing film PE film HyFol PP Plus, 16 μm (RKW Wasserburg, Germany). Apart from the test sample 1 produced, the measuring process should also include as a zero control and a negative control the carrying out of measurements with respect to the backing material as such and with respect to the backing material with the punched-out region covered over with adhesive tape, which should also be taken into consideration in the evaluation. The test samples and control samples are conditioned at 23° C. and 50% relative humidity for 24 hours and then measured at a differential pressure of 200 Pa on the basis of the method described at the beginning. For a comparative investigation of the air permeability, in an analogous way a corresponding test sample 2 is produced from the region surrounding the air-permeable region 350b and similarly subjected to measurement. Taking the control samples into consideration, the air-permeable region (test sample 1) has an air permeability of on average 121 l/m²/s; taking the control samples into consideration, the region surrounding this air-permeable region (test sample 2) has an air permeability of on average 48 l/m²/s (Table 1). The air-permeable region has comparatively an air permeability that is greater by a factor of 2.5. The openings that are introduced into the first region of the overhang and are essential to the invention have the effect that an incontinence article with improved wearing comfort is provided.

For comparative investigation of the water vapor permeability through the buildup of plies of the air-permeable region with the water vapor permeability of the buildup of plies likewise arranged in the first region of the overhang, but outside the air-permeable region, in an analogous way test sample 1 and test sample 2 and control samples are correspondingly produced, as described above for the measurement of the air permeability.

In the testing of the water vapor permeability, the test samples are introduced into the apparatus in such a way that the inner side that is assigned to the incontinence article in the form of briefs in the state of use, that is to say the side near the body, is facing in the direction of the inner side of the testing cup. As can be seen from the results from Table 2, the air-permeable region (test sample 1) has in comparison with the region surrounding this air-permeable region (test sample 2) a water vapor permeability that is greater by a factor of 2.3.

TABLE 1
Air permeability

| | Sample | Parameter | Unit | n | x | s | min | max |
|---|---|---|---|---|---|---|---|---|
| 1 | Intact backing | Test area | cm² | 1 | 20 | | 20 | 20 |
| | film | Air permeability | l/m²/s | 1 | 0 | | 0 | 0 |
| 2 | Backing film | Test area* | cm² | 2 | 3.09 | 0.01 | 3.08 | 3.10 |
| | with a punched- | Air permeability: value | l/m²/s | 2 | 0.45 | 0.09 | 0.39 | 0.52 |
| | out region of 7 × | read off** | | | | | | |
| | 45 mm | Air permeability with | l/m²/s | 2 | 2.92 | 0.57 | 2.52 | 3.32 |
| | covered over | respect to the actual | | | | | | |
| | with adhesive | surface area*** | | | | | | |
| | tape | | | | | | | |
| 3 | Test sample 2 | Test area* | cm² | 6 | 1.23 | 0.11 | 1.11 | 1.37 |
| | | Air permeability: value | l/m²/s | 6 | 3.12 | 1.00 | 1.50 | 4.00 |
| | | read off** | | | | | | |
| | | Air permeability with | l/m²/s | 6 | 50.39 | 14.66 | 25.00 | 65.81 |
| | | respect to the actual | | | | | | |
| | | surface area*** | | | | | | |
| 4 | Test sample 1 | Test area* | cm² | 6 | 1.39 | 0.21 | 1.17 | 1.64 |
| | | Air permeability: value | l/m²/s | 6 | 8.53 | 1.56 | 7.00 | 10.60 |
| | | read off** | | | | | | |
| | | Air permeability with | l/m²/s | 6 | 124.5 | 25.75 | 91.46 | 157.0 |
| | | respect to the actual | | | | | | |
| | | surface area*** | | | | | | |

*The test area corresponds to the surface area of the test piece that is effectively accessible after adhesively attaching the test piece in the carrier material
**The measured value read off relates to the defined size of the test area predetermined by the measuring apparatus, here 20 cm².
***The value standardized to the test area effectively used.
N Number of measurements
X Mean value
S Standard deviation
Min/max: Extreme values

TABLE 2
Water vapor permeability

| | Sample | Parameter | Unit | n | x | s | min | max |
|---|---|---|---|---|---|---|---|---|
| 1 | Zero control: | Weight A | g | 1 | 1160.24 | | 1160.24 | 1160.24 |
| | Intact backing | Weight B after | g | 1 | 1160.19 | | 1160.19 | 1160.19 |
| | film | 24 h | | | | | | |
| | | Area of dia. 62 mm | m² | 1 | 0.0030 | | 0.0030 | 0.0030 |
| | | WVTR | g/m²/24 h | 1 | 16.56 | | 16.56 | 16.56 |
| 2 | Backing film | Weight A | g | 1 | 1165.86 | | 1165.86 | 1165.86 |
| | with a punched- | Weight B after | g | 1 | 1165.82 | | 1165.82 | 1165.82 |
| | out region of 7 × | 24 h | | | | | | |
| | 45 mm | Sample area | m² | 1 | 0.00030 | | 0.00030 | 0.00030 |
| | covered over | WVTR | g/m²/24 h | 1 | 132.89 | | 132.89 | 132.89 |
| | with adhesive | | | | | | | |
| | tape | | | | | | | |
| 3 | Test sample 2 | Weight A | g | 6 | 1163.56 | 5.60 | 1155.59 | 1170.87 |
| | | Weight B after | g | 6 | 1163.28 | 5.62 | 1155.32 | 1170.63 |
| | | 24 h | | | | | | |
| | | Sample area^{#} | m² | 6 | 0.00016 | 0.00002 | 0.00014 | 0.00020 |
| | | WVTR | g/m²/24 h | 6 | 1669.47 | 169.03 | 1346.15 | 1829.27 |
| 4 | Test sample 1 | Weight A | g | 6 | 1161.71 | 7.44 | 1150.49 | 1172.08 |
| | | Weight B after | g | 6 | 1161.11 | 7.43 | 1149.94 | 1171.51 |
| | | 24 h | | | | | | |
| | | Sample area^{#} | m² | 6 | 0.00015 | 0.00002 | 0.00013 | 0.00018 |
| | | WVTR | g/m²/24 h | 6 | 3945.35 | 360.18 | 3475.61 | 4358.97 |

^{#}The test area corresponds to the surface area of the test piece that is effectively accessible after adhesively attaching the test piece in the carrier material
N Number of measurements
X Mean value
S Standard deviation
Min/max: Extreme values

1. An incontinence article (2) in the form of briefs for receiving body excretions, with a front stomach portion (4) and a rear back portion (6) which, to form a stomach and back band which is continuous in the transverse or waist-encircling direction (16) and has a waist opening (18) that is closed in the waist-encircling direction, are connected to one another at the manufacturer's at side seam regions (14) on both sides, and with a crotch portion (8), which has an absorbent body (7) and has longitudinal peripheries (48) and longitudinal ends (98, 100) and extends in a longitudinal direction (9) between the stomach portion (4) with a crotch-facing transverse periphery (58) and the back portion (6) with a crotch-facing transverse periphery (60), wherein the crotch portion (8) overlaps with the stomach portion (4) in a front overlapping region (36) and the crotch portion (8) overlaps with the back portion (6) in a rear overlapping region (38) while forming a respective buildup of plies (13) in the Z direction (11),
wherein the crotch portion (8) is inseparably joined to the stomach portion (4) in a front connecting region (306) and the crotch portion (8) is inseparably joined to the back portion (6) in a rear connecting region (308),
wherein not only the crotch portion (8) but also the stomach portion (4) and the back portion (6) bound leg openings (19) of the incontinence article,
wherein first elasticating means (28) are provided in the stomach portion (4) and the back portion (6), extend at a distance from one another and parallel to one another in the transverse or waist-encircling direction (16) and thus elasticate the stomach portion (4) and the back portion (6) over their surface area, wherein second elasticating means (40, 42) are provided in a crotch-side region (22, 26) of the stomach portion (4) and of the back portion (6) that is facing the leg openings (19),
wherein the crotch portion (8) comprises a liquid-impermeable backsheet material (62), and wherein the absorbent body (7) is arranged between the backsheet material (62) and a topsheet material (84) and the absorbent body (7) has longitudinal peripheries (46),
wherein the backsheet material (62) or the topsheet material (84) and the backsheet material (62) form in the transverse direction (16) an overhang (66a, 66b) extending respectively outside the longitudinal peripheries (46) of the absorbent body (7),
wherein the sum of the overhang (66a, 66b) in the transverse direction (16), that is to say on both sides of the longitudinal peripheries (46) of the absorbent body (7), is at least 25% with respect to the greatest width (E) of the crotch portion (8),
wherein the overhang (66a, 66b) on both sides of the longitudinal peripheries (46) of the absorbent body (7) respectively has first regions (65a, 65a′, 65b, 65b′) and a second region (67a, 67b),
wherein the first regions (65a, 65a′, 65b, 65b) respectively extend within the overlapping regions (36, 38) and the second region (67a, 67b) extends in the longitudinal direction (9) between the crotch-facing transverse peripheries (58, 60) of the stomach portion (4) and back portion (6), wherein the connecting regions (306, 308) of the stomach portion (4) and of the back portion (6) respectively comprise a first joining region (310, 312) and second joining regions (314a, 314b, 316a, 316b),
wherein the first joining region (310, 312) extends at least in certain portions in the region of the absorbent body (7), and wherein the second joining regions (314a, 314b, 316a, 316b) are arranged at least within the first regions (65a, 65a′, 65b, 65b′) of the overhang (66a, 66b) of the crotch portion (8), so that the second joining regions (314a, 314b, 316a, 316b) form reinforcing regions (334a, 334b, 336a, 336b), and
wherein the first regions (65a, 65a′, 65b, 65b′) of the overhang (66a, 66b) have openings (346) extending through the buildup of plies (13) in the Z direction in the front or rear overlapping region (36, 38), the openings (346) forming air-permeable regions (350a, 350b, 351a, 351b).

2. The incontinence article of claim 1, wherein the first regions (65a, 65a′, 65b, 65b′) of the overhang (66a, 66b) in the front and rear overlapping region (36, 38) have openings (346) extending through the buildup of plies (13) in the Z direction.

3. The incontinence article of claim 1, wherein the openings (346) are arranged at least within the second joining regions (314a, 314b, 316a, 316b).

4. The incontinence article of claim 1, wherein the openings (346) are formed by joining means (340) in the form of welding locations, in particular ultrasonic welding locations, thermal welding locations and/or calender welding locations.

5. The incontinence article of claim 1, wherein the second joining region (314a, 314b, 316a, 316b) extends over a subregion (324a, 324b, 326a, 326b) of the overhang (66a, 66b) in the transverse direction (16) with a width P′ and/or the air-permeable regions (350a, 350b, 351a, 351b) extend over a subregion (352a, 352b, 353a, 353b) of the overhang (66a, 66b) in the transverse direction (16) with a width M′.

6. The incontinence article of claim 1, wherein the second joining region (314a, 314b, 316a, 316b) ends at a distance (AP) before the longitudinal periphery (46) of the absorbent body (7), and particularly is arranged from the longitudinal periphery (48) of the crotch portion (8).

7. The incontinence article of claim 1, wherein the air-permeable region (350a, 350b, 351a, 351b) ends at a distance (AM) before the longitudinal periphery (46) of the absorbent body (7), and particularly is arranged from the longitudinal periphery (48) of the crotch portion (8).

8. The incontinence article of claim 1, wherein the respective second joining region (314a, 314b, 316a, 316b) and the respective air-permeable regions (350a, 350b, 351a, 351b) coincide in their areal extent, in particular are disposed congruently one on top of the other.

9. The incontinence article of claim 1, wherein a respective second joining region (314a, 314b, 316a, 316b), and consequently reinforcing region (334a, 334b, 336a, 336b) and/or a respective air-permeable region (350a, 350b, 351a, 351b) extends, when considered in the longitudinal direction (9), from the crotch-facing transverse periphery (58, 60) of the stomach portion (4) and/or of the back portion (6) in the direction of the longitudinal ends (98, 100) of the crotch portion (8), in particular extends continuously up to at least the respective longitudinal end (98, 100) of the crotch portion (8).

10. The incontinence article of claim 1, wherein a proportion P′/H of the subregion (324a, 324b, 326a, 326b) of the respective overhang (66a, 66b) that is extended over in the transverse direction (16) by a second joining region (314a, 314b, 316a, 316b), with a width (P′) with respect to the respective overhang (66a, 66b) in the transverse direction (16) with a width (H) in the front and/or rear overlapping region (36, 38), is at least 0.01, particularly at least 0.04, more particularly at least 0.07, more particularly at least 0.10, but preferably at most 0.90, particularly at most 0.80, more particularly at most 0.70, more particularly at most 0.60, more particularly at most 0.50, more particularly at most 0.40.

11. The incontinence article of claim 1, wherein a proportion M′/H of the subregion (352a, 352b, 353a, 353b) of the respective overhang (66a, 66b) that is extended over in the transverse direction (16) by an air-permeable region (350a, 350b, 351a, 351b), with a width M′ with respect to the respective overhang (66a, 66b) in the transverse direction (16) with a width H in the front and/or rear overlapping region (36, 38), is at least 0.01, particularly at least 0.04, more particularly at least 0.07, more particularly at least 0.10, but preferably at most 0.90, particularly at most 0.80, more particularly at most 0.70, more particularly at most 0.60, more particularly at most 0.50, more particularly at most 0.40.

12. The incontinence article of claim 1, wherein a proportion P′/N of the subregion (324a, 324b, 326a, 326b) of the respective overhang (66a, 66b) that is extended over in the transverse direction (16) by a second joining region (314a, 314b, 316a, 316b), with a width (P′) with respect to the respective side region (360a, 360b, 362a, 362b) of the stomach portion (4) and/or of the back portion (6) in the transverse direction (16) with a width (N), is at least 0.01, particularly at least 0.015, more particularly at least 0.020, but preferably at most 0.35, particularly at most 0.30, more particularly at most 0.25, more particularly at most 0.20, more particularly at most 0.15, more particularly at most 0.10.

13. The incontinence article of claim 1, wherein a proportion M′/N of the subregion (352a, 352b, 353a, 353b) of the respective overhang (66a, 66b) that is extended over in the transverse direction (16) by an air-permeable region (350a, 350b, 351a, 351b), with a width (M′) with respect to the respective side region (360a, 360b, 362a, 362b) of the stomach portion (4) and/or of the back portion (6) in the transverse direction (16) with a width (N), is at least 0.01, particularly at least 0.015, more particularly at least 0.020, but preferably at most 0.35, particularly at most 0.30, more particularly at most 0.25, more particularly at most 0.20, more particularly at most 0.15, more particularly at most 0.10.

14. The incontinence article of claim 1, wherein the openings (346) are arranged in a point pattern and wherein the sum of the surface area extended over by the openings (346) assumes a proportion of at least 1.5%, particularly at least 2.0%, more particularly at least 2.3%, more particularly at least 2.5% and preferably at most 20.0%, particularly at most 15.0%, more particularly at most 10.0%, more particularly at most 8.0%, more particularly at most 7.0%, more particularly at most 6.0%, with respect to the overall surface area extended over by an air-permeable region (350a, 350b, 351a, 351b), particularly by a second joining region (314a, 314b, 316a, 316b).

15. The incontinence article of claim 1, wherein the air-permeable region (350a, 350b, 351a, 351b) has an air permeability which is greater than the air permeability in the first region (65a, 65a′, 65b, 65b′) of the overhang (66a, 66b) outside the air-permeable region (350a, 350b, 351a, 351b) by a factor of 1.5, particularly 2, particularly 2.5, more particularly 3, more particularly at most 5, more particularly at most 4.5.

16. The incontinence article of claim 1, wherein the overhang (66a, 66b) of the backsheet material (62) and/or of the topsheet material (84) in the transverse direction (16) is in total, that is to say on both sides of the longitudinal peripheries (46) of the absorbent body (7), 25-50%, more particularly 30-45% and more particularly 35-45%, with respect to the greatest width (E) of the crotch portion (8).
